(19) United States
(12) Patent Application Publication
(10) Pub. No.: US 2024/0269246 A1
(43) Pub. Date: Aug. 15, 2024

(54) METHODS FOR TREATING CERVICAL DYSTONIA

(71) Applicant: REVANCE THERAPEUTICS, INC., Newark, CA (US)

(72) Inventors: Roman Rubio, Redwood City, CA (US); Thai Nguyen-Cleary, Fremont, CA (US); Chad Oh, Newark, CA (US)

(21) Appl. No.: 18/031,861

(22) PCT Filed: Oct. 13, 2021
(86) PCT No.: PCT/US2021/054818

§ 371 Date: Apr. 13, 2023

Related U.S. Application Data
(60) Provisional application No. 63/091,293, filed on Oct. 13, 2020.

(51) Int. Cl. A61K 38/48; A61K 9/00; A61K 47/42; A61P 21/02

(52) U.S. Cl. CPC A61K 38/4893; A61K 9/0019; A61K 47/42; A61P 21/02

(57) ABSTRACT

This invention provides methods to treat or prevent cervical dystonia, a disorder related thereto, or a symptom thereof, with novel injectable compositions comprising botulinum toxin that may be administered to a subject suffering from such maledy. The injectable compositions and methods in which these compositions are used provide novel and advantageous treatments which result in high responder rates and long duration of effect, for example, a duration of effect for 24 weeks and longer.

### CROSS REFERENCE TO RELATED APPLICATIONS

This application claims the benefit of the filing date of U.S. Patent Application No. 63/091,293, filed on Oct. 13, 2020, which is hereby incorporated herein by reference in its entirety.

### SEQUENCE LISTING

The instant application contains a Sequence Listing which has been submitted electronically in ASCII format and is hereby incorporated by reference in its entirety. Said ASCII copy, created on Oct. 13, 2021, is named 13720_0034_P_SL and is 7 Kilo bytes in size.

### FIELD OF THE INVENTION

This invention relates to methods to treat cervical dystonia, a disorder related thereto, or a symptom thereof, with novel injectable compositions comprising botulinum toxin that may be administered to a subject suffering from such maledy. The injectable compositions and methods in which these compositions are used provide novel and advantageous treatments, which result in high responder rates and long duration of effect, for example, a duration of effect for over 20 weeks.

### BACKGROUND OF THE INVENTION

Cervical dystonia is an extremely painful, chronic neurological movement disorder where the neck and shoulder muscles contract involuntarily and contort, causing abnormal movements and awkward posture of the head and neck such as the head to twist or turn to the left or right (torticollis), upwards (retrocollis), downwards (antecollis) or sideways (laterocollis). The movements may be sustained (tonic), jerky (clonic), or a combination. Cervical dystonia (also referred to as neck dystonia or spasmodic torticollis) affects a person's ability to control muscle activity. Cervical dystonia may be primary (meaning that it is the only apparent neurological disorder, with or without a family history) or may be brought about by secondary causes (such as physical trauma) and is often attributed to nervous system damage caused by a stroke, disease or trauma. A rare disorder that can occur at any age, even during infancy, cervical dystonia most often occurs in middle-aged individuals, and is more prevalent in women than men. Those with a family history of cervical dystonia or some other type of dystonia are at higher risk of developing the disorder.

Cervical dystonia is the third most common movement disorder following essential tremor and Parkinson's disease. An estimated 3 in every 10,000 people are known to suffer from cervical dystonia. The number of cases reported in North America alone is approximately 300,000.

Symptoms generally begin gradually and then reach a plateau where the symptoms don't get substantially worse. Unfortunately, there is no cure for cervical dystonia and the condition greatly impacts an individual's quality of life. In some cases, the disorder resolves without treatment, but sustained remissions are fairly uncommon.

The type A form of botulinum toxin is reported to be the most lethal natural biological agent known to man. Spores of C. botulinum are found in soil and can grow in improperly sterilized and sealed food containers. Botulism, which may be fatal, may be caused by the ingestion of the bacteria. Botulinum toxin acts to produce paralysis of muscles by preventing synaptic transmission by inhibiting the release of acetylcholine across the neuromuscular junction, and is thought to act in other ways as well. Its action essentially blocks signals that normally would cause muscle spasms or contractions, resulting in paralysis. During the last decade, botulinum toxin's muscle paralyzing activity has been harnessed to achieve a variety of therapeutic effects. Controlled administration of botulinum toxin has been used to provide muscle paralysis to treat a variety of medical conditions, for example, neuromuscular disorders characterized by hyperactive skeletal muscles. Conditions that have been treated with botulinum toxin include hemifacial spasm, adult onset spasmodic torticollis, anal fissure, blepharospasm, cerebral palsy, cervical dystonia, migraine headaches, strabismus, temporomandibular joint disorder, and various types of muscle cramping and spasms. More recently, the muscle-paralyzing effects of botulinum toxin have been applied to therapeutic and cosmetic facial applications such as treatment of wrinkles, frown lines, and other results of spasms or contractions of facial muscles.

In addition to the type A form of botulinum toxin, there are seven other serologically distinct forms of botulinum toxin that are also produced by the gram-positive bacteria Clostridium botulinum. Of these eight serologically distinct types of botulinum toxin, the seven that can cause paralysis have been designated botulinum toxin serotypes A, B, C, D, E, F and G. Each of these is distinguished by neutralization with type-specific antibodies. The molecular weight of each of the botulinum toxin proteins is about 150 kD. Due to the molecule size and molecular structure of botulinum toxin, it cannot cross stratum corneum and the multiple layers of the underlying skin architecture. The different serotypes of botulinum toxin vary in the effect and in the severity and duration of the paralysis they evoke in different animal species. For example, in rats, it has been determined that botulinum toxin type A is 500 times more potent than botulinum toxin type B, as measured by the rate of paralysis. Additionally, botulinum toxin type B has been determined to be non-toxic in primates at a dose of 480 U/kg, about 12 times the primate LD₅₀ for type A.

As released by Clostridium botulinum bacteria, botulinum toxin is a component of a toxin complex containing the approximately 150 kD botulinum toxin protein molecule along with associated non-toxin proteins. These endogenous non-toxin proteins are believed to include a family of hemagglutinin proteins, as well as non-hemagglutinin protein. The non-toxin proteins have been reported to stabilize the botulinum toxin molecule in the toxin complex and protect it against denaturation by digestive acids when toxin complex is ingested. Thus, the non-toxin proteins of the toxin complex protect the activity of the botulinum toxin and thereby enhance systemic penetration when the toxin complex is administered via the gastrointestinal tract. Additionally, it is believed that some of the non-toxin proteins specifically stabilize the botulinum toxin molecule in blood.

The presence of non-toxin proteins in the toxin complexes typically causes the toxin complexes to have molecular weights that are greater than that of the bare botulinum toxin molecule, which is about 150 kD, as previously stated. For example, Clostridium botulinum bacteria can produce botulinum type A toxin complexes that have molecular weights of about 900 kD, 500 kD or 300 kD. Botulinum toxin types B and C are produced as complexes having a molecular weight of about 700 kD or about 500 kD. Botulinum toxin type D is produced as complexes having molecular weights of about 300 kD or 500 kD. Botulinum toxin types E and F are only produced as complexes having a molecular weight of about 300 kD.

To provide additional stability to botulinum toxin, the toxin complexes are conventionally stabilized by combining the complexes with albumin during manufacturing. For example, BOTOX® (Allergan, Inc., Irvine, CA) is a botulinum toxin-containing formulation that contains 100 U of type A botulinum toxin with accessory proteins, 0.5 milligrams of human albumin, and 0.9 milligrams of sodium chloride. The albumin serves to bind and to stabilize toxin complexes in disparate environments, including those associated with manufacturing, transportation, storage, and administration.

Typically, the botulinum toxin is administered to patients by carefully controlled injections of compositions containing botulinum toxin complex and albumin. However, there are several problems associated with this approach. Not only are the injections painful, but typically large subdermal wells of toxin are locally generated around the injection sites, in order to achieve the desired therapeutic or cosmetic effect. The botulinum toxin may migrate from these subdermal wells to cause unwanted paralysis in surrounding areas of the body. This problem is exacerbated when the area to be treated is large and many injections of toxin are required to treat the area. Moreover, because the injected toxin complexes contain non-toxin proteins and albumin that stabilize the botulinum toxin and increase the molecular weight of the toxin complex, the toxin complexes have a long half-life in the body and may cause an undesirable antigenic response in the patient. For example, some patients will, over time, develop an allergy to the albumin used as a stabilizer in current commercial formulations. Also, the toxin complexes may induce the immune system of the patient to form neutralizing antibodies, so that larger amounts of toxin are required in subsequent administrations to achieve the same effect. When this happens, subsequent injections must be carefully placed so that they do not release a large amount of toxin into the bloodstream of the patient, which could lead to fatal systemic poisoning, especially since the non-toxin proteins and albumin stabilize the botulinum toxin in blood.

In view of the drawbacks associated with current botulinum toxin formulations, it would be highly desirable to have an injectable botulinum toxin formulation that is efficacious and stable, but exhibits reduced antigenicity and a lower tendency to diffuse locally after injection. It would also be desirable to use such a botulinum toxin formulation for therapeutic purposes to treat cervical dystonia.

Treatments for cervical dystonia include oral medications, botulinum toxin injections, surgery, and complementary therapies. The most commonly prescribed treatment for cervical dystonia is the use of botulinum toxin, typically type A (although Type B has also been used), which can reduce its signs and symptoms. Botulinum toxin can help block the communication between the nerve and the muscle and may alleviate abnormal movements and postures. The number of injections is typically based on the severity of the dystonia. Doctors injecting the toxin may select the muscles to be injected by observing abnormal postures or movements and feeling for the muscle spasm or by using an electromyography machine to measure muscle activity. Each muscle affected by dystonia typically has to be injected separately. As such, based on the diffusion characteristics of currently available toxin formulations, there is a limit to the total quantity of toxin that can be injected into the body at one time. While the treatment for cervical dystonia involves regular neurological intervention, which takes effect over a period of 4-7 days or longer after injection, the response to the treatment with botulinum toxin typically wears off after a 12 week period, often as early as at 10 weeks, requiring the person suffering from cervical dystonia to be injected again. Therefore, a durable, longer acting treatment requiring fewer neurological interventions would be desirable.

### SUMMARY OF THE INVENTION

In one of its aspects, the invention relates to a method for producing a biologic effect in the treatment of cervical dystonia by injecting an effective amount, preferably a therapeutically effective amount, of the compositions of this invention to a subject or patient in need of such treatment to reduce adverse events, such as dysphagia, muscle weakness, and/or musculoskeletal pain.

In another of its aspects, this invention provides a method of treating cervical dystonia in an individual in need thereof, the method comprising administering to the individual an injection of a composition comprising: a botulinum toxin, a botulinum toxin complex, or a reduced botulinum toxin complex and a positively charged carrier, such as one comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from 0 to 20; and a pharmaceutically acceptable diluent for injection; wherein the botulinum toxin is administered to the individual in an amount from about 100 U to about 500 U; wherein the one or more muscles comprise at least one of sternocleidomastoid, levator scapulae, and scalenus complex and wherein the sternocleidomastoid, levator scapulae, and scalenus complex are only unilaterally injected, and wherein the therapeutic effect is associated with a risk of adverse events, risk of dysphagia, risk of musculoskeletal pain, and/or risk of muscular weakness that is below 15%. The treatment can have at least about a 16 week, 20 week, 24 week, or 28 week duration of effect in reducing the symptoms of cervical dystonia, thereby extending treatment interval duration for the individual.

In another of its aspects, this invention provides a method of treating cervical dystonia in an individual in need thereof, the method comprising administering to the individual an injection of a composition comprising: a botulinum toxin, a botulinum toxin complex, or a reduced botulinum toxin complex and a positively charged carrier, such as one comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from 0 to 20; and a pharmaceutically acceptable diluent for injection; wherein the botulinum toxin is administered to the individual in an amount from about 100 U to about 500 U; wherein the one or more muscles are selected from a group comprising or consisting of splenius capitis, splenius cervices, trapezius, longissimus, sternocleidomastoid, levator scapulae, and scalenus complex, wherein the dose range for each of the one or more muscles is set forth in Table 1 or Table 6; and wherein the therapeutic effect is associated with a risk of adverse events, risk of dysphagia, risk of musculoskeletal pain, and/or risk of muscular weakness that is below 15%. The treatment can have at least about a 16 week, 20 week, 24 week, or 28 week duration of effect in reducing the symptoms of cervical dystonia, thereby extending treatment interval duration for the individual.

In another of its aspects, this invention provides injectable compositions comprising botulinum toxin non-covalently associated with a positively charged carrier molecule used to treat cervical dystonia. In preferred embodiments, the compositions of the invention possess one or more advantages over commercial botulinum toxin formulations, such as BOTOX®, XEOMINX®, DYSPORT®, or MYOBLOC®. For instance, in certain embodiments, the composition exhibits a longer duration of action and/or a lower incidence of adverse events as compared to commercial botulinum toxin formulations. In addition, one or more advantages over conventional injectable botulinum formulations can include reduced antigenicity, a reduced tendency to undergo diffusion into surrounding tissue following injection, increased duration of clinical efficacy or enhanced potency relative to conventional botulinum toxin formulations, faster onset of clinical efficacy, and/or improved stability.

A further aspect of this invention is the recognition that certain non-native molecules (i.e., molecules not found in botulinum toxin complexes obtained from Clostridium botulinum bacteria) can be added to botulinum toxin, botulinum toxin complexes, and in particular reduced botulinum toxin complexes (as defined herein), to improve toxin diffusion through tissues in the treatment of cervical dystonia. The non-native molecules associate non-covalently with the toxin and act as penetration enhancers that improve the ability of the toxin to reach target structures after injection. Furthermore, the non-native molecules may increase the stability of the toxin prior to and after injection. By way of example, the penetration enhancers may be positively charged carriers, such as cationic peptides, which have no inherent botulinum-toxin-like activity and which also contain one or more protein transduction domains as described herein.

Another aspect of this invention is to provide a composition comprising botulinum toxin, a botulinum toxin complex (or a reduced protein botulinum toxin complex including just the 150 kD neurotoxin itself, or the neurotoxin with some, but not all, of the native complex proteins) and a positively charged carrier for use in a method of treatment for cervical dystonia.

In another aspect, the invention provides effective doses and amounts of the compositions of this invention in the treatment of cervical dystonia that afford a long-lasting, sustained efficacy e.g., a response rate of long duration, following administration by injection to a subject or patient in need of treatment. Such doses and amounts are preferably therapeutically effective doses and amounts that produce or result in a desired therapeutic effect in a subject to whom the doses and amounts are administered. In particular embodiments, a single treatment of a subject or patient with a composition of the invention comprising a botulinum toxin, such as botulinum toxin A, and a positively charged carrier, as described herein, in therapeutically effective dose amounts of about 100 U to about 500 U per subject, afforded a response rate of in the reduction of cervical dystonia symptoms for at least 16 weeks, at least 20 weeks, at least 24 weeks, or about 6 to 10 months, or even longer. Moreover, the compositions of the invention provide an attribute of reduced diffusion or spread from the injection site following injection, thereby localizing the toxin and its effect where desired and decreasing nonspecific or unwanted effects of the toxin at sites or locations distant from the site of injection for treatment.

The duration of effect provided by compositions of the invention, e.g., RT002 as well as by the described treatment methods and uses, affords significant advantages compared to the art. By way of example, subjects undergoing treatment with compositions containing botulinum toxin consider that duration of effect following treatment is of high importance to them. Such a long, sustained duration of effect, which is achieved by even a single treatment with an effective dose and treatment regime of a product of the invention, for example, RT002, permits fewer injections per treatment course for a subject, which is extremely important for the subject. A prolonged duration effect from a single treatment with a product which has clear efficacy and safety, as provided by the inventive compositions and methods described herein, offer less discomfort, less cost and more convenience to subjects undergoing a course of treatment. Furthermore, a product that affords significant and sustained effects, which are maintained for at least a 16 or 24 week period, or for at least a 6-month period, or for greater than a 6-month period, following the single injectable treatment of the product to a subject, provides a solution to an unmet need in the art for both practitioners and patients alike. Thus, the compositions and methods of the invention provide a solution to the problem of too frequent treatments and improve patients' overall well-being. Such prolonged duration of action provides for fewer treatments over an entire treatment course.

In another aspect, the invention provides a method of administering botulinum toxin to achieve an extended duration therapeutic effect in an individual suffering from cervical dystonia, in which the method comprises administering by injection a dose of a sterile injectable composition into an area of the individual in need of treatment to achieve the therapeutic effect following a first treatment with the composition; wherein the composition comprises a botulinum toxin, a botulinum toxin complex, or a reduced botulinum toxin complex component and a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from 0 to 20; wherein the botulinum toxin, botulinum toxin complex, or reduced botulinum toxin complex component is administered to the individual in a treatment dose of about 100 U to 450 U; or more specifically, from about 100 U to 200 U (e.g., 125 U) or from about 200 U to 300 U (e.g., 250 U) or from about 300 U to 450 U, wherein the positively charged carrier is non-covalently associated with the botulinum toxin, botulinum toxin complex, or reduced botulinum toxin complex component; and a pharmaceutically acceptable diluent suitable for injection; and wherein the first treatment dose of the composition administered by injection to the individual achieves the extended duration therapeutic effect having at least a 16 week, 18 week, 20 week, 22 week, 24 week, 26 week, or 28 week duration of effect, optionally, before a second or subsequent treatment dose is administered.

In another aspect, the invention provides for the treatment of cervical dystonia, a sterile injectable composition comprising a botulinum toxin, a botulinum toxin complex, or a reduced botulinum toxin complex in a dosage amount selected from more than 100 U, from 100 U to 200 U (e.g., 125 U), 200 U to 300 U (e.g., 250 U), or 300-450 U; and a positively charged carrier comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from 0 to 20; and a pharmaceutically acceptable diluent for injection; wherein the positively charged carrier is non-covalently associated with the botulinum toxin, botulinum toxin complex, or reduced botulinum toxin complex component; and wherein the composition provides a therapeutic effect which endures for at least 16 to 20 weeks, 20 to 24 weeks, or for at least 6 months, or greater than 6 months, e.g., about 6 months to about 10 months, following a treatment of an individual with an effective dose of the injectable composition. In embodiments, the polylysine backbone has between 5 and 30 lysines or 10 to 20 lysines.

In some embodiments of these above methods and composition, the composition comprises botulinum toxin of serotype A, preferably a serotype A botulinum toxin having a molecular weight of 150 kDa. In an embodiment, the positively charged carrier has the amino acid sequence RKKRRQRRRG-(K)₁₅-GRKKRRQRRR (SEQ ID NO: 4). In an embodiment, the botulinum toxin is present in the composition in a dosage amount from more than 100 U, 100 U to 200 U (e.g., 125 U), 200 U to 300 U (e.g., 250 U), 300-450 U. In an embodiment, the botulinum toxin is present in the composition in a dosage amount selected from the group consisting of 100 U, 200 U, 300 U and 450 U. In an embodiment, the composition reduces the symptoms of cervical dystonia in an individual who has undergone a single treatment by injection of the composition. In certain embodiments, the duration of the treatment effect comprises greater than 6 months; greater than 7 months; greater than 8 months; greater than 9 months; or at least 6 months through 10 months.

In another of its aspects, the invention provides a method of treating an individual suffering from cervical dystonia who is in need of treatment with injectable botulinum toxin, in which the method of treatment comprises a treatment course having multiple treatment intervals with prolonged duration of effect and duration time between each treatment interval, the treatment course comprising: administering by injection an initial treatment dose of a sterile injectable composition into an area of the individual in need of treatment to achieve a therapeutic effect following the initial treatment with the composition; wherein the composition comprises a botulinum toxin, a botulinum toxin complex, or a reduced botulinum toxin complex component and a positively charged carrier component comprising a positively charged polylysine backbone having covalently attached thereto one or more positively charged efficiency groups having an amino acid sequence of (gly)p-RGRDDRRQRRR-(gly)q (SEQ ID NO: 1), (gly)p-YGRKKRRQRRR-(gly)q (SEQ ID NO: 2) or (gly)p-RKKRRQRRR-(gly)q (SEQ ID NO: 3), wherein the subscripts p and q are each independently an integer of from 0 to 20; and a pharmaceutically acceptable diluent suitable for injection; wherein the botulinum toxin, botulinum toxin complex, or reduced botulinum toxin complex component is administered to the individual in a treatment dose of more than 100 U, 125 U, 150 U, 175 U, 200 U, 225 U, 250 U, 175 U, 300 U, 325 U, 350 U, 375 U, 400 U, 425 U, 450 U, 500 U, or any value therebetween; wherein the positively charged carrier is non-covalently associated with the botulinum toxin, botulinum toxin complex, or reduced botulinum toxin complex component; wherein the initial treatment dose of the composition administered by injection to the individual provides a therapeutic duration of effect lasting through at least about 6 months; and administering subsequent treatment doses of the composition by injection to the individual at treatment intervals comprising a duration of greater than or equal to 16-20 weeks, 20-24 weeks, 24 to 28 weeks, 28 to 32 weeks, or up to at least 10 months following the initial treatment dose or between each subsequent treatment dose.

In embodiments of the above-described treatment method, the therapeutic effect is treatment of the symptoms of cervical dystonia. In an embodiment, the composition comprises botulinum toxin of serotype A, preferably, botulinum toxin of serotype A having a molecular weight of 150 kDa. In an embodiment, the positively charged carrier is a positively charged peptide having the amino acid sequence RKKRRQRRRG-(K)15-GRKKRRQRRR (SEQ ID NO: 4). In an embodiment, the composition does not locally diffuse from the site of injection following injection. In an embodiment, the botulinum toxin is present in the composition in a dosage amount more than or selected from the group consisting of 100 U, 125 U, 150 U, 175 U, 200 U, 225 U, 250 U, 175 U, 300 U, 325 U, 350 U, 375 U, 400 U, 425 U and 450 U. In certain embodiments, the duration of the treatment interval or the amount of time until a subsequent dose after a prior dose is a period between 16-20 weeks, 20-24 weeks, 24 to 28 weeks, or 28 to 32 weeks.

### DESCRIPTION OF THE FIGURES

FIG. 1 shows the Cervical Dystonia Phase 3 ASPEN-1 Study Design as described in Example 2 herein.

FIGS. 2A and 2B show ASPEN-1 demographics by cohort (10A) and baseline characteristics (10B) as described in Example 2. Percentages are based on the number of subjects in the intent-to-treat population. BoNT=botulinum neurotoxin.

FIG. 3 depicts injectable muscles as defined by the ASPEN-1 Study Design.

FIGS. 4A and 4B shows Primary Endpoint by Cohort—TWSTRS Total Score analysis of covariance of average at week 4 and 6 change from baseline in the Study as described in Example 3 herein (A, bar graph, B, tabular format). Statistics presented are based on an ANCOVA model with terms for treatment, pooled study center (region), prior BoNT treatment experience, and with Baseline TWSTRS total score as a covariate. Multiple imputation is used for subjects missing both Week 4 and Week 6 (n=3). *Least Square Means. Δ Change from baseline.

FIG. 5 shows Kaplan-Meier Plot of Duration of Effect. CI; confidence interval. Duration of effect is the time in weeks from treatment until loss of at least 80% of the peak treatment effect. The peak treatment effect is defined as the average change from baseline at week 4 and week 6 in the TWSTRS total score. Percentages are based on the number of subjects in the intent-to-treat population.

FIG. 6 shows comparison of duration of treatment effect by definition: Prespecified: Median Time to Loss of ≥80% of Peak Treatment Effect; Prespecified+Retreatment: Median Time to Prespecified Definition or Request for Retreatment; Prespecified+Retreatment w/ Responders: Median Time to Prespecified Definition or Request for Retreatment for Subjects Demonstrating Improvement at Primary Endpoint

FIG. 7 shows Secondary Endpoint-Responder at week 4 and 6 Patient Global Impression of Change (PGIC). Responder is defined as a response of moderately better (+2) or very much better (+3) at Week 4 or at Week 6. Missing responses for subjects still on study at Week 4 are considered as about the same (0). Δ Difference vs Placebo. P-value from a Cochran-Mantel-Haenszel test.

FIG. 8 shows Secondary Endpoint-Responder at week 4 and 6 Clincial Global Impression of Change (CGIC). Responder is defined as a response of moderately better (+2) or very much better (+3) at Week 4 or at Week 6. Missing responses for subjects still on study at Week 4 are considered as about the same (0). A Difference vs Placebo. P-value from a Cochran-Mantel-Haenszel test.

FIG. 9 shows Exploratory Endpoint-Average of the change from baseline in TWSTRS Subscale Scores at weeks 4 and 6. *Least square means. ΔChange from baseline.

FIG. 10 shows Secondary Endpoint-Change from baseline in TWSTRS-Total Score over time. Statistics plotted are observed Mean change from baseline in TWSTRS-Total Score. Data presented in the table are number of subjects with TWSTRS assessment at each visit.

FIG. 11 shows Exploratory Analysis-Change from baseline in TWSTRS-Total Score over time data for subjects who exit study are imputed with baseline score through week 36. Statistics plotted are observed mean change from baseline in TWSTRS-Total Score where subjects who exit the study are imputed at baseline score through week 36.

FIG. 12 is a plot comparing the duration of therapeutic effect observed with the currently available products according to the prescribing labels (Year 2020) as compared to the two RT002 doses by the amount of toxin in nanograms.

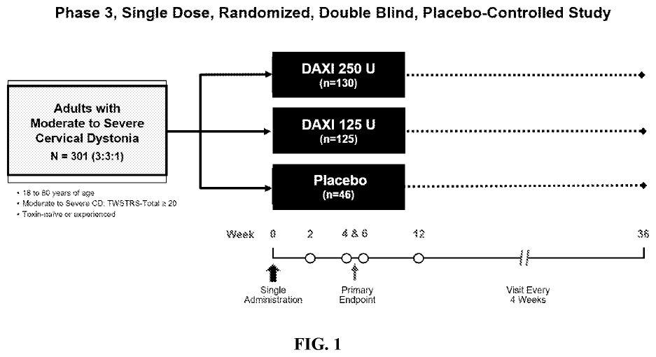

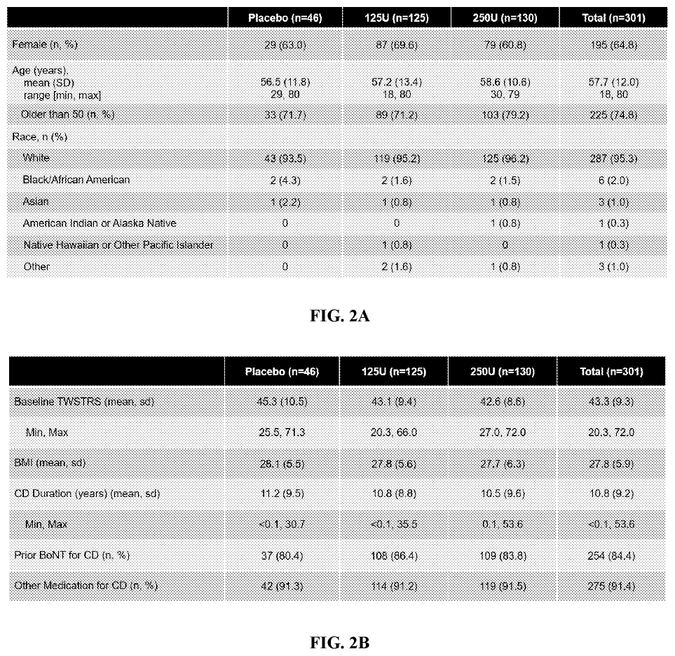

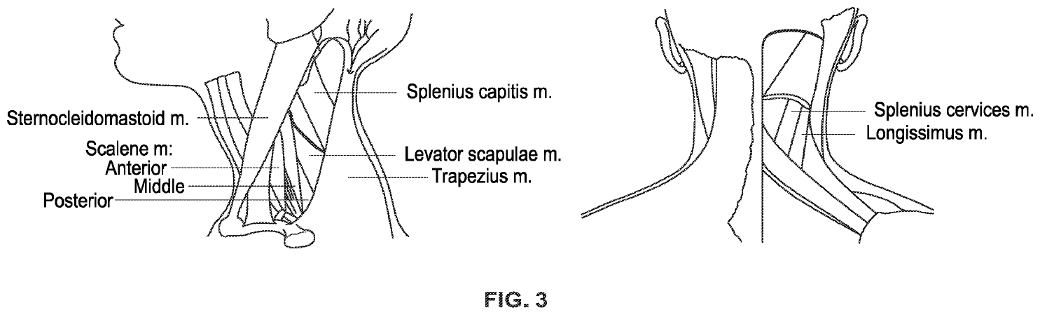

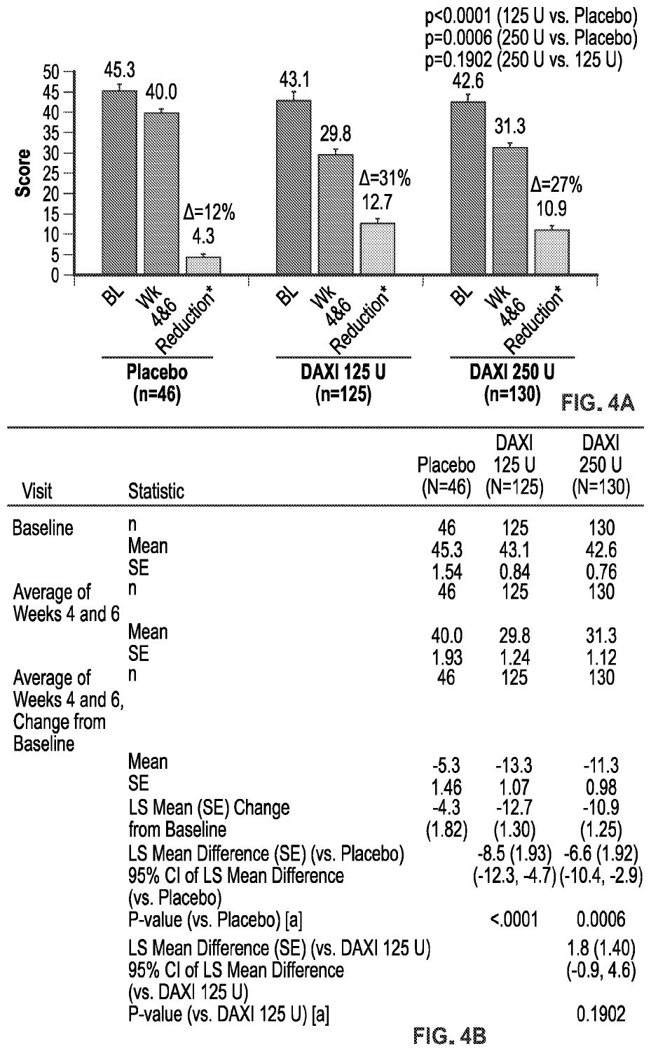

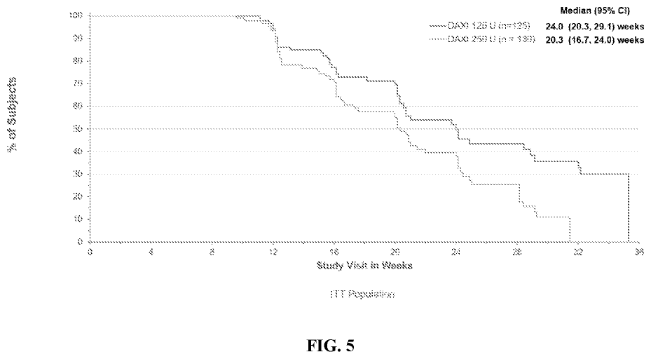

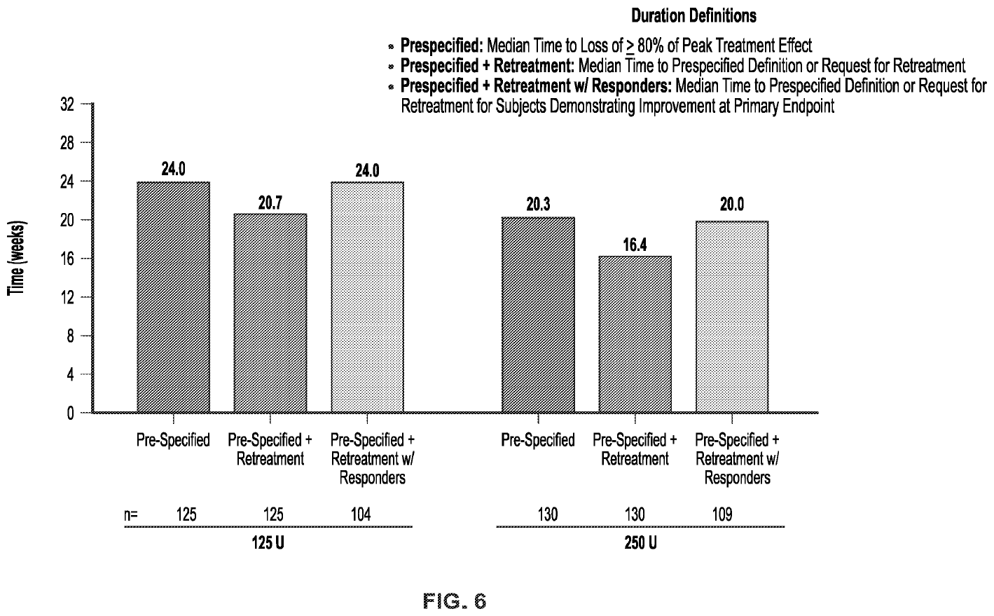

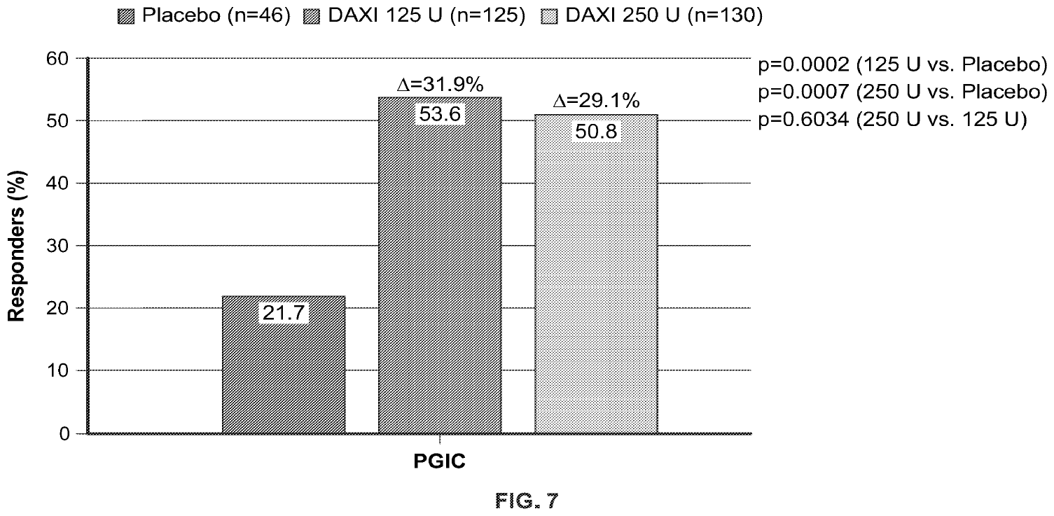

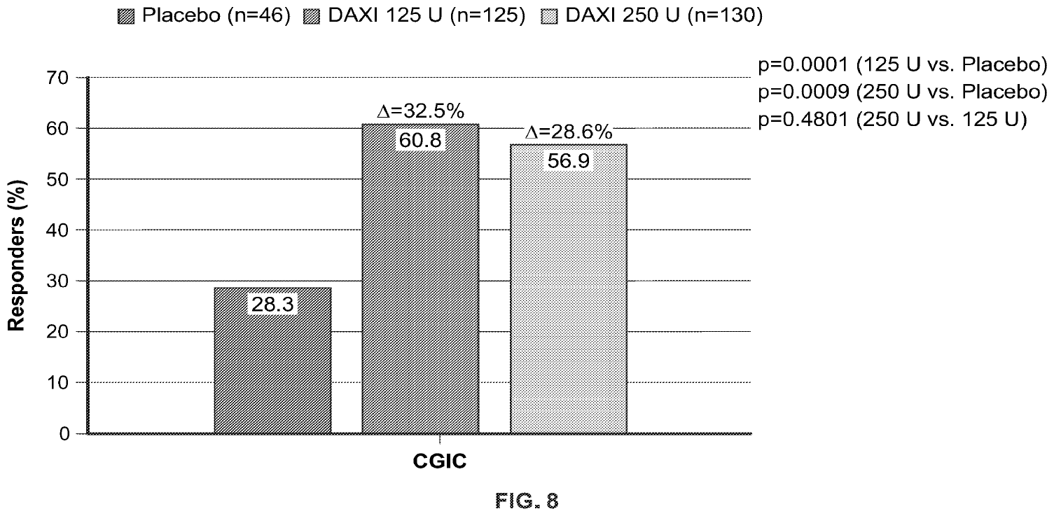

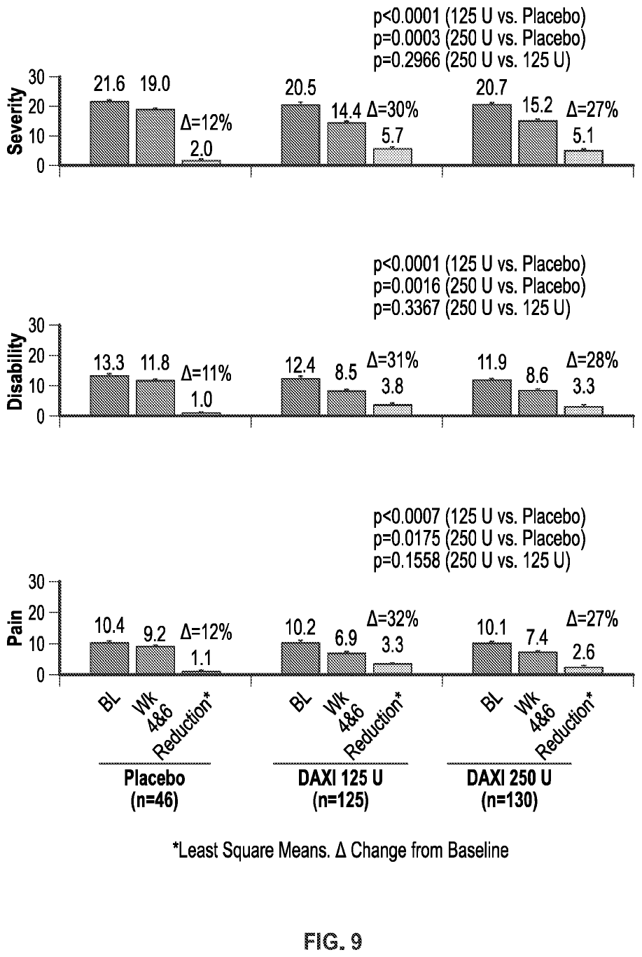

### DETAILED DESCRIPTION OF THE INVENTION

This invention relates to novel injectable compositions comprising botulinum toxin, a botulinum toxin complex, or a reduced botulinum toxin complex used in method to treat cervical dystonia. In preferred embodiments, the compositions stabilize the toxin or enable the transport or delivery of toxin through tissues after injection such that the toxin has reduced antigenicity, a better safety profile, enhanced potency, faster onset of clinical efficacy and/or longer duration of clinical efficacy compared to conventional commercial botulinum toxin complexes that are bound to exogenous albumin (e.g., BOTOX® or MYOBLOC®). The compositions of the invention may be used as injectable applications for providing a botulinum toxin to a subject, for various therapeutic, purposes, as described herein. The compositions of the invention also have an improved safety profile, including fewer adverse events, over other compositions and methods of delivery of botulinum toxin. In addition, these compositions can afford beneficial reductions in immune responses to the botulinum toxin. In embodiments, the injectable compositions of the invention provide long lasting efficacy, e.g., an effect lasting at least 16 weeks, 18 weeks, 20 weeks, or 24 weeks in subjects to whom such compositions, particularly those comprising botulinum toxin in amounts of 100 U to 500 U, are administered by injection for the treatment of cervical dystonia.

The term “botulinum toxin” as used herein may refer to any of the known types of botulinum toxin (e.g., 150 kD botulinum toxin protein molecules associated with the different serotypes of C. botulinum), whether produced by the bacterium or by recombinant techniques, as well as any such types that may be subsequently discovered including newly discovered serotypes, and engineered variants or fusion proteins. As mentioned above, currently seven immunologically distinct botulinum neurotoxins have been characterized, namely botulinum neurotoxin serotypes A, B, C, D, E, F and G, each of which is distinguished by neutralization with type-specific antibodies. The botulinum toxin serotypes are commercially available, for example, from Sigma-Aldrich (St. Louis, MO) and from Metabiologics, Inc. (Madison, WI), as well as from other sources. The different serotypes of botulinum toxin vary in the animal species that they affect and in the severity and duration of the paralysis they evoke. At least two types of botulinum toxin, types A and B, are available commercially in formulations for treatment of certain conditions. Type A, for example, is contained in preparations of Allergan having the trademark BOTOX® and of Ipsen having the trademark DYSPORT®, and type B is contained in preparations of Elan having the trademark MYOBLOC®.

The term “botulinum toxin” used in the compositions of this invention can alternatively refer to a botulinum toxin derivative, that is, a compound that has botulinum toxin activity but contains one or more chemical or functional alterations on any part or on any amino acid chain relative to naturally occurring or recombinant native botulinum toxins. For instance, the botulinum toxin may be a modified neurotoxin that is a neurotoxin which has at least one of its amino acids deleted, modified or replaced, as compared to a native form, or the modified neurotoxin can be a recombinantly produced neurotoxin or a derivative or fragment thereof. For instance, the botulinum toxin may be one that has been modified in a way that, for instance, enhances its properties or decreases undesirable side effects, but that still retains the desired botulinum toxin activity. Alternatively the botulinum toxin used in this invention may be a toxin prepared using recombinant or synthetic chemical techniques, e.g. a recombinant peptide, a fusion protein, or a hybrid neurotoxin, for example prepared from subunits or domains of different botulinum toxin serotypes (See, U.S. Pat. No. 6,444,209, for instance). The botulinum toxin may also be a portion of the overall molecule that has been shown to possess the necessary botulinum toxin activity, and in such case may be used per se or as part of a combination or conjugate molecule, for instance a fusion protein. Alternatively, the botulinum toxin may be in the form of a botulinum toxin precursor, which may itself be non-toxic, for instance a non-toxic zinc protease that becomes toxic on proteolytic cleavage.

The term “botulinum toxin complex” or “toxin complex” as used herein refers to the approximately 150 kD botulinum toxin protein molecule (belonging to any one of botulinum toxin serotypes A-G), along with associated endogenous non-toxin proteins (i.e., hemagglutinin protein and non-toxin non-hemagglutinin protein produced by Clostridium botulinum bacteria). Note, however, that the botulinum toxin complex need not be derived from Clostridium botulinum bacteria as one unitary toxin complex. For example, botulinum toxin or modified botulinum toxin may be recombinantly prepared first and then subsequently combined with the non-toxin proteins. Recombinant botulinum toxin can also be purchased (e.g., from List Biological Laboratories, Campbell, CA) and then combined with non-toxin proteins.

This invention also contemplates modulation of the stability of botulinum toxin molecules through the addition of one or more exogenous stabilizers, the removal of endogenous stabilizers, or a combination thereof. For example, this invention contemplates the use of “reduced botulinum toxin complexes”, in which the botulinum toxin complexes have reduced amounts of non-toxin protein compared to the amounts naturally found in botulinum toxin complexes produced by Clostridium botulinum bacteria. In one embodiment, reduced botulinum toxin complexes are prepared using any conventional protein separation method to extract a fraction of the hemagglutinin protein or non-toxin non-hemagglutinin protein from botulinum toxin complexes derived from Clostridium botulinum bacteria. For example, reduced botulinum toxin complexes may be produced by dissociating botulinum toxin complexes through exposure to red blood cells at a pH of 7.3 (e.g., see EP 1514556 A1, hereby incorporated herein by reference). HPLC, dialysis, columns, centrifugation, and other methods for extracting proteins from proteins can be used. Alternatively, when the reduced botulinum toxin complexes are to be produced by combining synthetically produced botulinum toxin with non-toxin proteins, one may simply add less hemagglutinin or non-toxin, non-hemagglutinin protein to the mixture than what would be present for naturally occurring botulinum toxin complexes. Any of the non-toxin proteins (e.g., hemagglutinin protein or non-toxin non-hemagglutinin protein or both) in the reduced botulinum toxin complexes according to the invention may be reduced independently by any amount. In certain exemplary embodiments, one or more non-toxin proteins are reduced by at least about 0.5%, 1%, 3%, 5%, 10%, 20%, 30%, 40%, 50%, 60%, 70%, 80%, 90% or 100% compared to the amounts normally found in botulinum toxin complexes. As noted above, Clostridium botulinum bacteria produce seven different serotypes of toxin and commercial preparations are manufactured with different relative amounts of non-toxin proteins (i.e. different amount of toxin complexes). For example, MYOBLOC™ has 5000 U of Botulinum toxin type B per ml with 0.05% human serum albumin, 0.01 M sodium succinate, and 0.1 M sodium chloride. DYSPORT™ has 500 U of botulinum toxin type A-hemagglutinin complex with 125 mcg albumin and 2.4 mg lactose. In certain embodiments, substantially all of the non-toxin protein (e.g., greater than 95%, 96%, 97%, 98% or 99% of the hemagglutinin protein and non-toxin non-hemagglutinin protein) that would normally be found in botulinum toxin complexes derived from Clostridium botulinum bacteria is removed from the botulinum toxin complex. Furthermore, although the amount endogenous non-toxin proteins may be reduced by the same amount in some cases, this invention also contemplates reducing each of the endogenous non-toxin proteins by different amounts, as well as reducing at least one of the endogenous non-toxin proteins, but not the others.

As noted above, an exogenous stabilizer (e.g., albumin) is typically added to stabilize botulinum toxin formulations. For instance, in the case of BOTOX®, 0.5 mg of human albumin per 100 U of type A botulinum toxin complex to stabilize the complex. Generally, the amount of exogenous stabilizer that may be added to stabilize the compositions according to the invention is not particularly limited. In some embodiments, the amount of added stabilizer may be less than the amount conventionally added, owing to the ability of positively charged carriers of the invention to act as a stabilizer in its own right. For instance, the amount of added exogenous albumin can be any amount less than the conventional thousand-fold excess of exogenous albumin and, in certain exemplary embodiments of the invention, is only about 0.25, 0.20, 0.15, 0.10, 0.01, 0.005, 0.001, 0.0005, 0.00001, 0.000005, 0.000001, or 0.0000001 mg per 100 U of botulinum toxin. In one embodiment, no exogenous albumin is added as a stabilizer to the compositions of the invention, thus producing albumin-free botulinum toxin compositions.

A preferred composition of the invention is a liquid, botulinum toxin-containing composition that is stabilized without a proteinaceous excipient, especially without any animal protein-derived excipients. Such a liquid composition comprises a botulinum toxin, preferably botulinum toxin of serotype A, a positively charged carrier (e.g., peptide) a non-reducing disaccharide or a non-reducing trisaccharide, a non-ionic surfactant, and a physiologically compatible buffer for maintaining the pH between 4.5. and 7.5. The concentration of the non-reducing sugar in the liquid composition is in the range of 10% through 40% (w/v) and the concentration of the non-ionic surfactant is in the range of 0.005% through 0.5% (w/v). The preferred composition provides a long duration effect after treatment by a single injection In a preferred embodiment, the botulinum toxin A has a molecular weight (MW) of 150 kDa. The preferred composition comprises botulinum toxin, preferably botulinum toxin A, more preferably, of 150 kDa MW, a positively charged carrier (e.g., peptide) as described herein, a non-reducing disaccharide, such as sucrose, a non-ionic surfactant, such as polysorbate 20, polysorbate 40, polysorbate 60, polysorbate 80, or a sorbitan ester, and a physiologically compatible buffer, such as citric acid, acetic acid, succinic acid, tartaric acid, maleic acid, and histidine; and has a pH in the range of pH 4.5. to pH 7.5.

According to the present invention, a positively charged carrier molecule having protein transduction domains or efficiency groups, as described herein, has been found suitable as a transport system for a botulinum toxin, enabling toxin to be injected with improved penetration to target structures such as muscles. The transport occurs without covalent modification of the botulinum toxin. Besides enhancing penetration of botulinum toxin, the positively charged carriers of the invention may, in certain preferred embodiments, stabilize the botulinum toxin against degradation. In such embodiments, the hemagglutinin protein and non-toxin, non-hemagglutinin protein that are normally present to stabilize the botulinum toxin may be reduced or omitted entirely. Similarly, the exogenous albumin that is normally added during manufacturing may be omitted.

By the use of the terms “positively charged” or “cationic” in connection with the term “carrier”, it is meant that the carrier has a positive charge under at least some solution-phase conditions, more preferably, under at least some physiologically compatible conditions. More specifically, “positively charged” and “cationic” as used herein, means that the group in question contains functionalities that are charged under all pH conditions, for instance, a quaternary amine, or contains a functionality which can acquire positive charge under certain solution-phase conditions, such as pH changes in the case of primary amines. More preferably, “positively charged” or “cationic” as used herein refers to those groups that have the behavior of associating with anions over physiologically compatible conditions. Polymers with a multiplicity of positively-charged moieties need not be homopolymers, as will be apparent to one skilled in the art. Other examples of positively charged moieties are well known in the prior art and can be employed readily, as will be apparent to those skilled in the art.

Generally, the positively-charged carrier (also referred to as a “positively charged backbone”) is typically a chain of atoms, either with groups in the chain carrying a positive charge at physiological pH, or with groups carrying a positive charge attached to side chains extending from the backbone. In certain preferred embodiments, the positively charged backbone is a cationic peptide. As used herein, the term “peptide” refers to an amino acid sequence, but carries no connotation with respect to the number of amino acid residues within the amino acid sequence. Accordingly, the term “peptide” may also encompass polypeptides and proteins. In certain preferred embodiments, the positively charged backbone itself will not have a defined enzymatic or therapeutic biologic activity. In certain embodiments, the backbone is a linear hydrocarbon backbone which is, in some embodiments, interrupted by heteroatoms selected from nitrogen, oxygen, sulfur, silicon and phosphorus. The majority of backbone chain atoms are usually carbon. Additionally, the backbone will often be a polymer of repeating units (e.g., amino acids, poly(ethyleneoxy), poly(propyleneamine), polyalkyleneimine, and the like) but can be a heteropolymer. In one group of embodiments, the positively charged backbone is a polypropyleneamine wherein a number of the amine nitrogen atoms are present as ammonium groups (tetra-substituted) carrying a positive charge. In another embodiment, the positively charged backbone is a nonpeptidyl polymer, which may be a hetero- or homo-polymer such as a polyalkyleneimine, for example a polyethyleneimine or polypropyleneimine, having a molecular weight of from about 10,000 to about 2,500,000, preferably from about 100,000 to about 1,800,000, and most preferably from about 500,000 to about 1,400,000. In another group of embodiments, the backbone has attached a plurality of side-chain moieties that include positively charged groups (e.g., ammonium groups, pyridinium groups, phosphonium groups, sulfonium groups, guanidinium groups, or amidinium groups). The sidechain moieties in this group of embodiments can be placed at spacings along the backbone that are consistent in separations or variable. Additionally, the length of the sidechains can be similar or dissimilar. For example, in one group of embodiments, the sidechains can be linear or branched hydrocarbon chains having from one to twenty carbon atoms and terminating at the distal end (away from the backbone) in one of the above-noted positively charged groups. The association between the positively charged carrier and the botulinum toxin is by non-covalent interaction, non-limiting examples of which include ionic interactions, hydrogen bonding, van der Waals forces, or combinations thereof. Examples of positively charged backbones and efficiency groups for use of the invention are described in U.S. Pat. Nos. 8,623,811 and 9,211,248, which are hereby incorporated by reference in their entirety.

In one group of embodiments, the positively charged backbone is a polypeptide having multiple positively charged sidechain groups (e.g., lysine, arginine, ornithine, homoarginine, and the like). Preferably, the polypeptide has a molecular weight from about 100 to about 1,500,000, more preferably from about 500 to about 1,200,000, most preferably from about 1000 to about 1,000,000. One of skill in the art will appreciate that when amino acids are used in this portion of the invention, the sidechains can have either the D- or L-form (R or S configuration) at the center of attachment. In certain preferred embodiments, the polypeptide has a molecular weight from about 500 to about 5000, more preferably from 1000 to about 4000, more preferably from 2000 to about 3000. In other preferred embodiments, the polypeptide comprises 10 to 20 amino acids, or 10, 11, 12, 13, 14, 15, 16, 17, 18, 19, or 20 amino acids, preferably polylysine.

Alternatively, the backbone may comprise amino acid analogs and/or synthetic amino acids. The backbone may also be an analog of a polypeptide such as a peptoid. See, for example, Kessler, Angew. Chem. Int. Ed. Engl. 32:543 (1993); Zuckermann et al. Chemtracts-Macromol. Chem. 4:80 (1992); and Simon et al. Proc. Nat'l. Acad. Sci. USA 89:9367 (1992)). Briefly, a peptoid is a polyglycine in which the sidechain is attached to the backbone nitrogen atoms rather than the α-carbon atoms. As above, a portion of the sidechains will typically terminate in a positively charged group to provide a positively charged backbone component.

Synthesis of peptoids is described in, for example, U.S. Pat. No. 5,877,278, which is hereby incorporated by reference in its entirety. As the term is used herein, positively charged backbones that have a peptoid backbone construction are considered “non-peptide” as they are not composed of amino acids having naturally occurring sidechains at the alpha-carbon locations.

A variety of other backbones can be used employing, for example, steric or electronic mimics of polypeptides wherein the amide linkages of the peptide are replaced with surrogates such as ester linkages, thioamides (—CSNH—), reversed thioamide (—NHCS—), aminomethylene (—NHCH₂—) or the reversed methyleneamino (—CH₂NH—) groups, keto-methylene (—COCH₂—) groups, phosphinate (—PO₂RCH₂—), phosphonamidate and phosphonamidate ester (—PO₂RNH—), reverse peptide (—NHCO—), trans-alkene (—CR═CH—), fluoroalkene (—CF═CH—), dimethylene (—CH₂CH₂—), thioether (—CH₂S—), hydroxyethylene (—CH(OH)CH₂—), methyleneoxy (—CH₂O—), tetrazole (CN₄), sulfonamido (—SO₂NH—), methylenesulfonamido (—CHRSO₂NH—), reversed sulfonamide (—NHSO₂—), and backbones with malonate and/or gem-diamino-alkyl subunits, for example, as reviewed by Fletcher et al. ((1998) Chem. Rev. 98:763) and detailed by references cited therein. Many of the foregoing substitutions result in approximately isosteric polymer backbones relative to backbones formed from α-amino acids.

In each of the backbones provided above, sidechain groups can be appended that carry a positively charged group. For example, the sulfonamide-linked backbones (—SO₂NH— and —NHSO₂—) can have sidechain groups attached to the nitrogen atoms. Similarly, the hydroxyethylene (—CH(OH)CH₂—) linkage can bear a sidechain group attached to the hydroxy substituent. One of skill in the art can readily adapt the other linkage chemistries to provide positively charged sidechain groups using standard synthetic methods.

In one embodiment, the positively charged backbone is a polypeptide having protein transduction domains (also referred to as efficiency groups). As used herein, an efficiency group or protein transduction domain is any agent that has the effect of promoting the translocation of the positively charged backbone through a tissue or cell membrane. Non-limiting examples of protein transduction domains or efficiency groups include -(gly)ₙ₁-(arg)ₙ₂ (SEQ ID NO: 5), HIV-TAT or fragments thereof, or the protein transduction domain (PTD) of Antennapedia, or a fragment thereof, in which the subscript n1 is an integer of from 0 to 20, more preferably 0 to 8, still more preferably 2 to 5, and the subscript n2 is independently an odd integer of from about 5 to about 25, more preferably about 7 to about 17, most preferably about 7 to about 13. In some embodiments, the HIV-TAT fragment does not contain the cysteine-rich region of the HIV-TAT molecule, in order to minimize the problems associated with disulfide aggregation. Preferably, the fragments of the HIV-TAT and Antennapedia protein transduction domains retain the protein transduction activity of the full protein. Still further preferred are those embodiments in which the HIV-TAT fragment has the amino acid sequence (gly)ₚ-RGRDDRRQRRR-(gly)_{q} (SEQ ID NO: 1), (gly)ₚ-YGRKKRRQRRR-(gly)_{q} (SEQ ID NO: 2) or (gly)ₚ-RKKRRQRRR-(gly)_{q} (SEQ ID NO: 3) wherein the subscripts p and q are each independently an integer of from 0 to 20, or wherein p and q are each independently the integer 1. In another embodiment, the fragment or efficiency group is attached to the backbone via either the C-terminus or the N-terminus of the fragment or amino acid sequence of the efficiency group. In certain preferred embodiments, p is one and q is zero or p is zero and q is one. Preferred HIV-TAT fragments are those in which the subscripts p and q are each independently integers of from 0 to 8, more preferably 0 to 5. In another preferred embodiment the positively charged side chain or branching group is the Antennapedia (Antp) protein transduction domain (PTD), or a fragment thereof that retains activity. These are known in the art, for instance, from Console et al., J. Biol. Chem. 278:35109 (2003) and a non-limiting example of an Antennapedia PTD contemplated by this invention is the PTD having the amino acid sequence SGRQIKIWFQNRRMKWKKC (SEQ ID NO: 6). In other embodiments, the positively charged carrier is a positively charged peptide having the amino acid sequence RKKRRQRRR-G-(K)₁₅-G-RKKRRQRRR (SEQ ID NO: 4); or a positively charged peptide having the amino acid sequence YGRKKRRQRRR-G-(K)₁₅-G-YGRKKRRQRRR (SEQ ID NO: 7); or a positively charged peptide having the amino acid sequences RGRDDRRQRRR-G-(K)₁₅-G-RGRDDRRQRRR (SEQ ID NO: 8) for use in the compositions and methods of the invention.

Preferably the positively charged carrier includes side-chain positively charged protein transduction domains or positively charged efficiency groups in an amount of at least about 0.01%, as a percentage of the total carrier weight, preferably from about 0.01 to about 50 weight percent, more preferably from about 0.05 to about 45 weight percent, and most preferably from about 0.1 to about 30 weight %. For positively charged protein transduction domains having the formula -(gly)ₙ₁-(arg)ₙ₂ (SEQ ID NO: 5), a preferred range is from about 0.1 to about 25%.

In another embodiment, the backbone portion is a polylysine and positively charged protein transduction domains are attached to the lysine sidechain amino groups or to the C- or N-termini. In some preferred embodiments, the polylysine may have a molecular weight that is at least 100, 200, 300, 400, 500, 600, 700, 800, 900, 1000, 1500, 2000, 2500, 3000, 3500, 4000, 4500, 5000, 5500, or 6000 D, and less than about 2,000,000, 1,000,000, 500,000, 250,000, 100,000, 75,000, 50,000, and 25,000 D. Within the range of 100 to 2,000,000 D, it is contemplated that the lower and/or upper range may be increased or decreased, respectively, by 100, with each resulting sub-range being a specifically contemplated embodiment of the invention. In some exemplary embodiments, the polylysine has a molecular weight from about 1,000 to about 1,500,000 D, from about 2,000 to about 800,000 D, or from about 3,000 to about 200,000 D. In other exemplary embodiments, the polylysine has molecular weight from about 100 to about 10,000 D, from about 500 to about 5,000 D, from about 1,000 to about 4,000 D, from about 1,500 to about 3,500 D or from about 2,000 to about 3,000 D. Preferred is a polylysine polypeptide having 10 to 20 lysines (SEQ ID NO: 9), more preferably, 15 lysines. In some embodiments, the polylysine contemplated by this invention can be any of the commercially available (Sigma Chemical Company, St. Louis, Mo., USA) polylysines such as, for example, polylysine having MW>70,000, polylysine having MW of 70,000 to 150,000, polylysine having MW 150,000 to 300,000 and polylysine having MW>300,000. The selection of an appropriate polylysine will depend on the remaining components of the composition and will be sufficient to provide an overall net positive charge to the composition and provide a length that is preferably from one to four times the combined length of the negatively charged components. Preferred positively charged protein transduction domains or efficiency groups include, for example, -gly-gly-gly-arg-arg-arg-arg-arg-arg-arg (-Gly₃Arg₇ (SEQ ID NO: 10)) or HIV-TAT.

In another preferred embodiment the positively charged backbone is a polyalkyleneimine, non-limiting examples of which include polyethyleneimine, polypropyleneimine, and polybutyleneimine. In certain embodiments, the polyalkyleneimine has a molecular weight of at least 100, 200, 300, 400, 500, 600, 700, 800, 900, 1000, 1500, 2000, 2500, 3000, 3500, 4000, 4500, 5000, 5500, or 6000 D, and less than about 2,000,000, 1,000,000, 500,000, 250,000, 100,000, 75,000, 50,000, and 25,000 D. Within the range of 100 to 2,000,000 D, it is contemplated that the lower and/or upper range may be increased or decreased, respectively, by 100, with each resulting sub-range being a specifically contemplated embodiment of the invention.

In other embodiments of this invention, the carrier is a relatively short polylysine or polyethyleneimine (PEI) backbone (which may be linear or branched) and which has positively charged branching groups. Without wishing to be constrained by theory, it is believed that such carriers are useful for minimizing uncontrolled aggregation of the backbones and botulinum toxin in a therapeutic composition, which causes the transport efficiency to decrease dramatically. When the carrier is a relatively short linear polylysine or PEI backbone, the backbone will have a molecular weight of less than 75,000 D, more preferably less than 30,000 D, and most preferably, less than 25,000 D. When the carrier is a relatively short branched polylysine or PEI backbone, however, the backbone will have a molecular weight less than 60,000 D, more preferably less than 55,000 D, and most preferably less than 50,000 D.

In one particularly interesting embodiment, the non-native molecules are cationic peptides that have no inherent botulinum-toxin-like activity and that also contain one or more protein transduction domains as described herein. Without wishing to be bound by any particular scientific theory, it is believed that the peptides enhance tissue penetration of molecules associated in complex after injection, while enhancing stabilization of the botulinum toxin in skin and in vitro. It is believed that the enhanced tissue penetration afforded by these peptides in particular affords reduced antigenicity, a better safety profile, enhanced potency, faster onset of clinical efficacy or longer duration of clinical efficacy compared to conventional commercial botulinum toxin complexes that are bound to exogenous albumin (e.g., BOTOX® or MYOBLOC®).

In preferred embodiments, the concentration of positively charged carriers in the compositions according to the invention is sufficient to enhance the delivery of the botulinum toxin to molecular targets such as, for example, motor nerve plates. Furthermore, without wishing to be bound by theory, it is believed that the penetration rate follows receptor-mediated kinetics, such that tissue penetration increases with increasing amounts of penetration-enhancing-molecules up to a saturation point, upon which the transport rate becomes constant. Thus, in a preferred embodiment, the amount of added penetration-enhancing-molecules is equal to the amount that maximizes penetration rate right before saturation. A useful concentration range for the positively charged carrier (or carrier peptide) in the injectable compositions of this invention is about 0.1 pg of carrier per Unit (U) of botulinum toxin (0.1 pg/U) to about 1.0 mg per Unit (mg/U) of the botulinum toxin as described herein. A useful concentration range for the positively charged carrier (or carrier peptide) in the topical compositions of the invention is about 1.0 pg/U to 0.5 mg/U of botulinum toxin (amount of carrier/U of botulinum toxin). In other embodiments, the positively charged carrier (or carrier peptide) is present in the injectable compositions of the invention in the range of, for example, 1 ng/U to 300 ng/U or 10 ng/U to 200 ng/U of botulinum toxin, or in the range of 1 ng/U to 1000 ng/U of botulinum toxin; or in the range of 0.1 ng/U to 10,000 ng/U of botulinum toxin. In some embodiments, the amount of positively charged carrier (or carrier peptide) to Units of botulinum toxin present in the compositions of the invention is, by way of nonlimiting example, 10, 20, 30, 40, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200 ng of carrier per Unit of botulinum toxin (ng/U), or any amount therebetween. Preferably, the botulinum toxin is of serotype A, and particularly, the 150 kD form of serotype A botulinum toxin and the carrier peptide is that shown at SEQ ID NO: 4.

In general, methods and procedures for measuring the activity of botulinum toxin, i.e., units (U) of botulinum toxin activity, are known to and practiced by those having skill in the art. Briefly, median lethality assays (LD₅₀ assays) in mice are conventionally used to estimate the number of units of botulinum toxin with a high degree of precision. Doses of all commercially available botulinum toxins are expressed in terms of units of biologic activity. By way of example, one unit of botulinum toxin corresponds to the calculated median intraperitoneal lethal dose (LD50) in female Swiss-Webster mice. See, Hoffman, R. O. et al., 1986, Int. Ophthalmol. Clin., 26:241-50, as well as DePass, L. R., 1989, Toxicol. Letters, 49:159-170; and Pearce, L. B. et al., 1994, Toxicol. Appl. Pharmacol., 128:69-77, which also describe lethality assays in the art. More particularly, a suitable method for determining botulinum toxin units for a botulinum toxin component of the compositions of the invention is as follows: Forty-eight (48) female CD-1 mice weighing 17-23 grams are randomly assigned to six doses of the test article (1.54, 1.31, 1.11, 0.95, 0.80, and 0.68 U/0.5 mL), eight (8) animals per dose group. The test article refers to the botulinum toxin preparation or sample being assayed or tested. The animals are housed eight per cage and are weighed within 24 hours of dosing with the test article. On the day of dosing, the test article is diluted to the appropriate concentrations in isotonic saline (0.9% NaCl). Each animal is administered 0.5 mL of diluted test article via intraperitoneal injection. After injection, mice are returned to the cage and fatalities are recorded daily for three days. Lethality is scored 72 hours post injection and the results are analyzed by probit or logistic analysis to derive the LD₅₀ value relative to a reference standard that is assessed using the same dosing regimen. By way of example, the reference standard is a specifically qualified and calibrated lot of the same composition of the invention that is used for comparison to derive relative potency of the test article. The determined LD₅₀ value is then corrected for the cumulative dilutions performed to assign a relative potency value for the neat (undiluted) test article.

Compositions of this invention are preferably in a form that permits injection into the skin or epithelium of subjects or patients. The term “in need” is meant to include both pharmaceutical or health-related needs (e.g., treating conditions involving undesirable dystonic contractions or muscle spasms). In preferred embodiments, the compositions are prepared by mixing the botulinum toxin (either containing the associated non-toxin proteins or reduced associated non-toxin proteins) with the positively charged carrier, and usually with one or more additional pharmaceutically acceptable carriers or excipients. In their simplest form, they may contain an aqueous pharmaceutically acceptable diluent, such as buffered saline (e.g., phosphate buffered saline). However, the compositions may contain other ingredients typically found in injectable pharmaceutical or cosmeceutical compositions, including a dermatologically or pharmaceutically acceptable carrier, vehicle or medium that is compatible with the tissues to which it will be applied. The term “pharmaceutically acceptable,” as used herein, means that the compositions or components thereof so described are suitable for use in contact with these tissues or for use in patients in general without undue toxicity, incompatibility, instability, allergic response, and the like. As appropriate, compositions of the invention may comprise any ingredient conventionally used in the fields under consideration.

In terms of their form, compositions of this invention may include solutions, emulsions (including microemulsions), suspensions, gels, powders, or other typical solid or liquid compositions used for injection to muscle and other tissues where the compositions may be used. In preferred embodiments, the compositions of the invention are present in low-viscosity, sterile formulations suitable for injection with a syringe. As used herein, the terms compositions and formulations are essentially interchangeable when referring to the compositions and formulations according to the present invention. The compositions of the invention may be in the form of a lyophilized powder that is reconstituted using a pharmaceutically acceptable liquid diluent prior to injection. In certain embodiments, the lyophilized powder is reconstituted with a liquid diluent to form an injectable formulation with a viscosity of about 0.1 to about 2000 cP, more preferably about 0.2 to about 500 cP, even more preferably about 0.3 to about 50 cP, and even more preferably about 0.4 to about 2.0 cP. The compositions of the invention may contain, in addition to the botulinum toxin and positively charged carrier, other ingredients typically used in such products, such as antimicrobials, hydration agents, tissue bulking agents or tissue fillers, preservatives, emulsifiers, natural or synthetic oils, solvents, surfactants, detergents, gelling agents, antioxidants, fillers, thickeners, powders, viscosity-controlling agents and water, and optionally including anesthetics, anti-itch actives, botanical extracts, conditioning agents, minerals, polyphenols, silicones or derivatives thereof, vitamins, and phytomedicinals.

The injectable compositions according to this invention may be in the form of controlled-release or sustained-release compositions which comprise botulinum toxin and positively charged carrier encapsulated or otherwise contained within a material such that they are released within the tissue in a controlled manner over time. The composition comprising the botulinum toxin and positively charged carrier may be contained within matrixes, liposomes, vesicles, microcapsules, microspheres and the like, or within a solid particulate material, all of which is selected and/or constructed to provide release of the botulinum toxin over time. The botulinum toxin and the positively charged carrier may be encapsulated together (i.e., in the same capsule) or separately (i.e., in separate capsules).

In embodiments, compositions of the invention comprise liquid (aqueous) compositions (or formulations) comprising a botulinum toxin as described herein, a positively charged carrier (or peptide) as described herein, a non-reducing disaccharide or a non-reducing trisaccharide, a non-ionic surfactant, and a physiologically compatible buffer, which is capable of maintaining a suitable pH, such as a pH in the range of pH 4.5 to pH 7.5, or pH 4.5 to pH 6.8, or pH 4.5 to pH 6.5. It is to be understood that a suitable pH also includes the upper and lower pH values in the range, e.g., a pH of 6.5 or a pH of 7.5. The concentration of the non-reducing sugar in the liquid composition is in the range of 10% through 40% (w/v) and the concentration of the non-ionic surfactant is in the range of 0.005% through 0.5% (w/v). The liquid compositions may be dried, preferably by lyophilization, to produce stabilized solid compositions, which may thereafter be reconstituted for use, for example, using sterile saline or other known physiologically and pharmaceutically acceptable diluents, excipients, or vehicles, especially those known for use in injectable formulations. Preferably, the dried, e.g., lyophilized, solid compositions are noncrystalline and amorphous solid compositions, and may be in the form of powders, for example. Also, preferably, the compositions of the invention do not include animal protein-derived products, such as albumin. Compositions that are suitable for the invention are also described in U.S. Pat. No. 9,340,587 B2, the entire contents of which are incorporated herein by reference. In particular embodiments the compositions comprise botulinum toxin of serotype A. In other particular embodiments, the compositions comprise botulinum toxin of serotype A which has a molecular weight of 150 kDa.

In certain embodiments, the compositions of the invention contain a non-reducing sugar, which is preferably a disaccharide, non-limiting examples of which include trehalose, including its anhydrous and hydrated forms, or sucrose, as well as combinations thereof. In some embodiments, the hydrated form of trehalose, trehalose-dihydrate, is preferable. In other embodiments, the compositions contain a trisaccharide, a non-limiting example of which is raffinose. In general, the concentration of the non-reducing sugar, preferably a disaccharide, e.g., sucrose, in the compositions of the invention are in the range of 10% to 40% (w/v), preferably 10% to 25% (w/v), more preferably 15% to 20% (w/v). In some preferred embodiments, the concentration of the non-reducing sugar, preferably a disaccharide, e.g., sucrose, is 10%, 11%, 12%, 13%, 14%, 15%, 16%, 17%, 18%, 19% or 20% (w/v).

In general, the compositions of the invention may include any non-ionic surfactant that has the ability to stabilize botulinum toxin and that is suitable for pharmaceutical use. In some embodiments, the non-ionic surfactant is a polysorbate, such as, by way of nonlimiting example, polysorbate 20, polysorbate 40, polysorbate 60, and polysorbate 80. In other embodiments, the non-ionic surfactant is a sorbitan ester, non-limiting examples of which include SPAN® 20, SPAN® 60, SPAN® 65, and SPAN® 80. The non-ionic surfactants Triton® X-100 or NP-40 may also be used. In addition, a combination of the different non-ionic surfactants may be used. In certain preferred embodiments, the non-ionic surfactant is a polysorbate, a poloxamer and/or a sorbitan; polysorbates and sorbitans are particularly preferred. In embodiments, the non-ionic surfactant is present in the compositions of the invention in the range of 0.005% to 0.5%, or in the range of 0.01% to 0.2%, or in the range of 0.02% to 0.1% or in the range of 0.05 to 0.08%, inclusive of the upper and lower values. In addition, the compositions of the invention may contain a non-ionic surfactant in the amount of 0.01%, 0.02%, 0.03%, 0.04%, 0.05%, 0.06%, 0.07%, 0.08%, 0.09%, 0.10%, 0.11%, 0.12%, 0.13%, 0.14%, or 0.15%.

In general for the compositions of the invention, any physiologically compatible buffer capable of maintaining the pH in the above ranges is suitable for use. Non-limiting examples of such buffers include salts of citric acid, acetic acid, succinic acid, tartaric acid, maleic acid, and histidine. Non-limiting examples of suitable buffer concentrations include buffer concentrations in the range of 0.400% to 0.600%; 0.450% to 0.575%, or 0.500% to 0.565%. The compositions of the invention may also comprise a mixture of buffer salts, non-limiting examples of which include citrate/acetate, citrate/histidine, citrate/tartrate, histidine monohydrochloride, maleate/histidine, or succinate/histidine. Accordingly, a composition of the invention which provides a long duration effect after treatment by a single injection includes a botulinum toxin, such as botulinum toxin A or botulinum toxin A of 150 kDa MW, as described herein, a positively charged carrier (or peptide) as described herein, a non-reducing disaccharide, such as sucrose, a non-ionic surfactant, such as polysorbate 20, polysorbate 40, polysorbate 60, polysorbate 80, or a sorbitan ester, and a physiologically compatible buffer, such as citric acid, acetic acid, succinic acid, tartaric acid, maleic acid, and histidine, which is capable of maintaining a suitable pH, such as a pH in the range of pH 4.5 to pH 6.5 or in the range of pH 4.5. to pH 7.5, in w/v amounts as described herein.

A particular composition of the invention is an albumin-free, liquid (aqueous) composition which comprises a botulinum toxin, preferably botulinum toxin of serotype A, or a botulinum toxin A having a molecular weight of 150 kDa; a positively charged carrier (e.g., peptide); a non-reducing disaccharide or a non-reducing trisaccharide, preferably a disaccharide, present in a range of 10% through 40% (w/v); a non-ionic surfactant, preferably, a polysorbate or sorbitan ester, present in the range of 0.005% through 0.5% (w/v); and a physiologically compatible buffer, such as citric acid, acetic acid, succinic acid, tartaric acid, maleic acid, or histidine, present in the range of 0.400% to 0.600%; 0.450% to 0.575%, or 0.500% to 0.565%, for maintaining the pH between 4.5. and 7.5.

Botulinum toxin formulations according to the invention can be delivered by injection (typically using a syringe) to muscles underlying the skin, or to glandular structures within the skin, in an effective amount to produce paralysis, produce relaxation, alleviate contractions, prevent or alleviate spasms, reduce glandular output, or other desired effects. Local delivery of the botulinum toxin in this manner could afford dosage reductions, reduce toxicity and allow more precise dosage optimization for desired effects relative to injectable or implantable materials.

The compositions of the invention are administered to deliver an effective amount, preferably a therapeutically effective amount, of the botulinum toxin. The term “effective amount” or “therapeutically effective amount” as used herein means an amount of a botulinum toxin as defined above that is sufficient to produce the desired muscular paralysis or other biological effect, but that implicitly is a safe amount, i.e., one that is low enough to avoid serious side effects.

The compositions of the invention may contain an appropriate effective amount of the botulinum toxin for application as a single-dose treatment, or may be more concentrated, either for dilution at the place of administration or for use in multiple applications and/or sequential applications over periods of time. Through the use of the positively charged carrier this invention, a botulinum toxin can be administered by injection to a subject for treating conditions such as cervical dystonia. The botulinum toxin is administered by injection to muscles or to other skin-associated or other target tissue structures.

Most preferably, the compositions are administered by or under the direction of a physician or other health care professional. They may be administered in a single treatment or in a series of treatments over time. In preferred embodiments, a composition according to the invention is injected at a location or locations where an effect associated with botulinum toxin is desired. In the treatment of cervical dystonia, the following Table 1 provides guidance as to the appropriate dosage of RT002 for injection where a dose is 125 U or 250 U, respectively:

TABLE 1
Injectable Muscles and Pre-defined Injection Volume by Muscle
Total volume of reconstituted study drug is 2.5 mL Dilution

| | | RT002 for | RT002 for |
|---|---|---|---|
| | | Injection 125 U | Injection 250 U |
| | | Group | Group |
| | Mandatory | 5 U/ 0.1 mL | 10 U/ 0.1 mL |
| | Injection Volume by | Dose Range | Dose Range |
| Injectable Muscles | Muscle (mL) | (Units) | (Units) |
| Sternocleidomastoid | 0.2-0.5 | 10-25 | 20-50 |
| Levator scapulae | 0.2-0.6 | 10-30 | 20-60 |
| Scalenus complex | 0.2-0.3 | 10-15 | 20-30 |
| Splenius capitis | 0.2-1.0 | 10-50 | 20-100 |
| Splenius cervices | 0.2-1.0 | 10-50 | 20-100 |
| Trapezius | 0.3-0.8 | 15-40 | 30-80 |
| Longissimus | 0.2-0.6 | 10-30 | 20-60 |

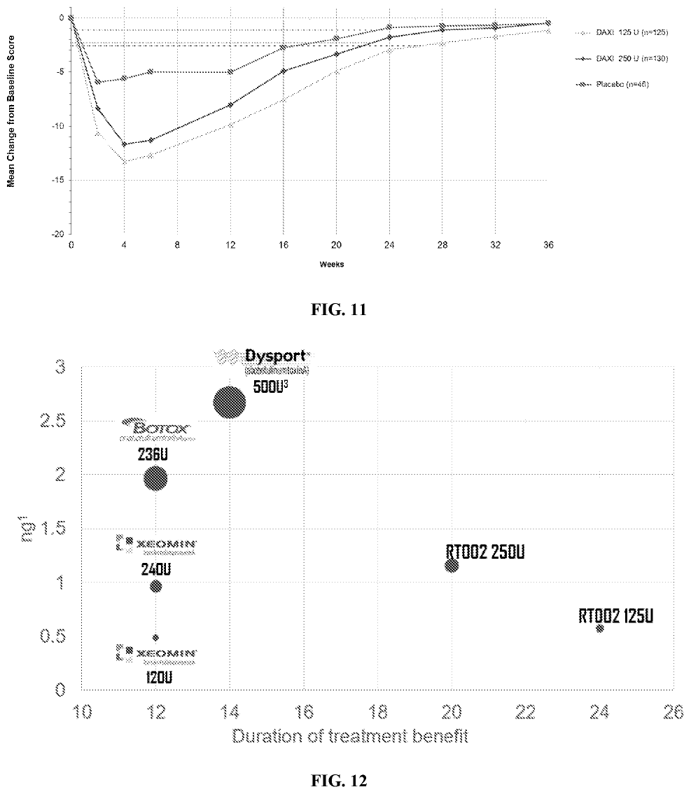

One or more muscles in the head, neck, and shoulder area are injected with the botulinum toxin composition. In some embodiments, the one or more muscular targets are selected from a group consisting of the sternocleidomastoid, levator scapulae, scalenus complex, splenius capitis, splenius cervices, trapezius, and longissimus, the left and right side muscle or muscle complex each being an optional target. (see FIG. 11). In some embodiments, 2, 3, 4, 5, 6, 7, 8, 9, 10, 11, 12, 13, or all muscular targets are selected from a group consisting of sternocleidomastoid, levator scapulae, scalenus complex, splenius capitis, splenius cervices, trapezius, and longissimus, the left and right side muscle or muscle complex each being an optional target. In some embodiments, the splenius capitis, splenius cervices, trapezius, and longissimus may receive unilateral or bilateral injections (i.e., injections in one or both sides of the body). In some embodiments, only unilateral injections are administered in the sternocleidomastoid, levator scapulae, and/or scalenus complex. In some embodiments, the sternocleidomastoid, levator scapulae, scalenus complex, splenius capitis, splenius cervices, trapezius, and longissimus each receive at least one injection. In some embodiments, a muscular target can receive one or more injections, such as 1, 2, 3, 4, 5, etc. In some embodiments, the dosage range administered to each muscular target, by one or more injections, is provided in Table 1.

Because of its nature, the botulinum toxin preferably is administered at an amount, application rate, and frequency that will produce the desired result without producing any adverse or undesired results. In embodiments, a single treatment with an effective dose of the compositions of the invention affords an effect of long duration such that during a course of treatment for an indication treatable by botulinum toxin, or series of injections during a single multiple treatment session, with a concomitant effect that endures over extended periods of time, e.g., at least 12. 14, 16, 18, 20, 24, 26, 28, 32, 36, and 38 weeks; or 3 months, 4 months, 5 months, 6 months or greater than 6 months, namely, 6 months, 7 months, 8 months, 9 months, or longer, including 10 months. The longer duration of action provides for longer intervals or time periods between treatments where multiple treatments are used to maintain a treatment goal or effect. In an embodiment, the longer duration of effect of the composition following administration to, or dosing of, an individual with a composition of the invention providing about 100 U to 500 U; or more specifically, from about 100 U, 125 U, 150 U, 175 U, 200 U, 225 U, 250 U, 175 U, 300 U, 325 U, 350 U, 375 U, 400 U, 425 U, 450 U, or any amount therebetween of botulinum toxin, for example, at least 12. 14, 16, 18, 20, 24, 26, 28, 32, 36, and 38 weeks or 3 months, 4 months, 5 months, 6 months or greater than 6 months, such as 7, 8, 9, or 10 months, including in between, is relative to a duration of effect of a botulinum toxin-containing composition or product that does not contain a positively charged carrier (or peptide) according to the present invention. In some cases, a composition or product containing botulinum toxin without a positively charged carrier (or peptide) of the invention is effective for less than 3 or 4 months. The results herein are preferably achieved through the use of the RT002 formulation as described below.

In certain embodiments, the compositions of the invention, which comprise a botulinum toxin and a positively charged carrier comprising a positively charged polymeric backbone with one or more covalently attached positively charged efficiency groups as described herein, are administered as a single injection to a subject or patient in need thereof in an amount or at a dose which provides about 100 U to 500 U; or more specifically, from about 100 U, 125 U, 150 U, 175 U, 200 U, 225 U, 250 U, 175 U, 300 U, 325 U, 350 U, 375 U, 400 U, 425 U, 450 U, or any amount therebetween of botulinum toxin per treatment dose per subject for the treatment of cervical dystonia. According to the invention, a treatment effect endures for several weeks or months, for example, for at least 10 weeks, for at least 12 weeks, for at least 16 weeks, for at least 20 weeks, for at least 24 weeks, for at least 28 weeks, for at least 30 weeks, or for at least 6 months, or greater than 6 months, such as 6, 7, 8, 9, or 10 months, or longer. In embodiments, the botulinum toxin is of serotype A, B, C, D, E, F, or G. In an embodiment, the botulinum toxin is of serotype A. In an embodiment, the serotype A botulinum toxin has a molecular weight of 150 kDa. In an embodiment, the serotype A botulinum toxin is in the form of a higher molecular weight complex as described supra. In preferred embodiments, the 150 kDa botulinum toxin or the higher molecular weight forms of the toxin are in albumin-free formulations. In an embodiment, the positively charged polymeric backbone is polylysine or polyethyleneimine. In an embodiment, the one or more positively charged efficiency groups include -(gly)ₙ₁-(arg)ₙ₂ (SEQ ID NO: 5), in which the subscript n1 is an integer of from 0 to 20, more preferably 0 to 8, still more preferably 2 to 5, and the subscript n2 is independently an odd integer of from about 5 to about 25, more preferably about 7 to about 17, most preferably about 7 to about 13. In some embodiments, the one or more positively charged efficiency groups has the amino acid sequence (gly)ₚ-RGRDDRRQRRR-(gly)_{q} (SEQ ID NO: 1), (gly)ₚ-YGRKKRRQRRR-(gly)_{q} (SEQ ID NO: 2) or (gly)ₚ-RKKRRQRRR-(gly)_{q} (SEQ ID NO: 3), wherein the subscripts p and q are each independently an integer of from 0 to 20. In certain preferred embodiments, p is one and q is zero or p is zero and q is one. In other preferred embodiments, the subscripts p and q are each independently integers of from 0 to 8, more preferably 0 to 5. In a particular embodiment, the positively charged carrier has the amino acid sequence RKKRRQRRRG-(K)₁₅-GRKKRRQRRR (SEQ ID NO: 4). In other embodiments, the one or more positively charged efficiency groups is attached to the positively charged backbone via either the C-terminus or the N-terminus of the efficiency group, e.g., amino acid sequence. In some embodiments, the one or more positively charged efficiency groups are attached to either end, or both ends, of the positively charged polylysine backbone of the positively charged carrier. In particular embodiments, the positively charged backbone is polylysine and the botulinum toxin is of serotype A. In another particular embodiment, the serotype A botulinum toxin has a molecular weight of 150 kDa, the positively charged backbone is polylysine and the one or more covalently attached positively charged efficiency groups has the amino acid sequence (gly)ₚ-RGRDDRRQRRR-(gly)_{q} (SEQ ID NO: 1), (gly)ₚ-YGRKKRRQRRR-(gly)_{q} (SEQ ID NO: 2) or (gly)ₚ-RKKRRQRRR-(gly)_{q} (SEQ ID NO: 3), wherein the subscripts p and q are each independently an integer of from 0 to 20, or are each independently the values as set forth above; or the positively charged carrier has the amino acid sequence RKKRRQRRRG-(K)₁₅-GRKKRRQRRR (SEQ ID NO: 4). In embodiments, the composition is administered by injection in an amount or dose that provides 50 U or at least 50 U; 60 U or at least 60 U; 70 U or at least 70 U; 80 U or at least 80 U; 90 U or at least 90 U; or 100 U or at least 100 U, 125 U or at least 125 U, 150 U or at least 150 U; 175 U or at least 175 U; 200 U or at least 200 U, 225 U or at least 225 U, 250 U or at least 250 U; 275 U or at least 275 U; 300 U or at least 300 U, 325 U or at least 325 U, 350 U or at least 350 U; 375 U or at least 375 U; 400 U or at least 400 U of botulinum toxin per injection. Amounts or doses between the foregoing amounts or doses are also contemplated, for example, 130 U or at least 10 3 U; 170 U or at least 170 U; 20 U or at least 210 U, and the like. In particular embodiments, the composition is administered by injection as a single treatment dose in an amount that provides about 100 U to 500 U; or more specifically, from about 100 U to 200 U or from about 200 U to 300 U or from about 300 U to 450 U, of botulinum toxin and a response or effect is achieved and maintained for a long duration, e.g., for at least 10 weeks, for at least 12 weeks, for at least 14 weeks, for at least 16 weeks, for at least 18 weeks, for at least 20 weeks, for at least 22 weeks, at least 24 weeks, at least 6 months, or greater than 6 months, such as, for example, 6, 7, 8, 9, or 10 months, or longer.

Without wishing to be limiting, in a course of treatment, the compositions of the invention may be administered at less frequent intervals following an initial treatment dose based on the extended duration of effect afforded by the therapeutically effective doses of the compositions and methods of the invention as described herein. For example, the compositions of the invention may be administered (or dosed) to an individual in need about twice per year (about every 6 months), or every 3 months, 4 months, 5 months, 7 months, by the practice of the methods of the invention. In a particular embodiment, an individual is administered a dose of a composition of the invention twice per year or three times per year or four times per year. A median duration between doses may be 3 months, 4 months, 5 months, 6 months, at least 6 months, or greater than 6 months, depending on the therapeutic treatment and/or the desire for treatment as determined by the individual being treated. A composition of the invention may be dosed at the appropriate interval at about 100 U to 500 U; or more specifically, at 100 U, 125 U, 150 U, 175 U, 200 U, 225 U, 250 U, 175 U, 300 U, 325 U, 350 U, 375 U, 400 U, 425 U, 450 U, or any amount therebetween of botulinum toxin in the composition.

This invention also contemplates the use of a variety of delivery devices for injecting botulinum toxin-containing compositions described herein across skin. Such devices may include, without limitation, a needle and syringe, or may involve more sophisticated devices capable of dispensing and monitoring the dispensing of the composition, and optionally monitoring the condition of the subject in one or more aspects (e.g., monitoring the reaction of the subject to the substances being dispensed).

In some embodiments, the compositions can be pre-formulated and/or pre-installed in a delivery device as such. This invention also contemplates embodiments wherein the compositions are provided in a kit that stores one or more components separately from the remaining components. For example, in certain embodiments, the invention provides for a kit that separately stores botulinum toxin and the positively charged carrier for combining at or prior to the time of application. The amount of positively charged carrier or the concentration ratio of these molecules to the botulinum toxin will depend on which carrier is chosen for use in the composition in question. The appropriate amount or ratio of carrier molecule in a given case can readily be determined, for example, by conducting one or more experiments such as those described below.

In general, the invention also contemplates a method for administering botulinum toxin (alternatively as botulinum toxin complexes or reduced botulinum toxin complexes) to a subject or patient in need thereof, in which an effective amount of botulinum toxin is administered in conjunction with a positively charged carrier, as described herein. By “in conjunction with” it is meant that the two components (botulinum toxin and positively charged carrier) are administered in a combination procedure, which may involve either combining them prior to administration to a subject, or separately administering them, but in a manner such that they act together to provide the requisite delivery of an effective amount of the therapeutic protein. For example, a composition containing the positively charged carrier may first be administered to the skin of the subject, followed by application a skin patch, syringe, or other device containing the botulinum toxin. The botulinum toxin may be stored in dry form in a syringe or other dispensing device and the positively charged carrier may be injected before application of the toxin so that the two act together, resulting in the desired tissue penetration enhancement. In that sense, thus, the two substances (positively charged carrier and botulinum toxin) act in combination or perhaps interact to form a composition or combination in situ. Accordingly, the invention also includes a kit with a device for dispensing botulinum toxin and a liquid, gel, or the like that contains the positively charged carrier, and that is suitable for injection to the skin or target tissue of a subject. Kits for administering the compositions of the inventions, either under direction of a health care professional or by the patient or subject, may also include a custom applicator suitable for that purpose.

The compositions of this invention are suitable for use in physiologic environments with pH ranging from about 4.5 to about 6.3, and may thus have such a pH. However, compositions having a pH ranging from about 4.5 to about 7.5 are also embraced by the invention as described herein. The compositions according to this invention may be stored either at room temperature or under refrigerated conditions.

In some embodiments, the patient to be treated is 65 years of age, at least 65 years old, or over 65 years old. For example, the patient may be 65, 66, 68, 70, 75, 80 years, or older.

### EXAMPLES

### Example 1—Formulation

An injectable botulinum toxin formulation (RT002) was prepared with ingredients according to Table 2 below. All inactive ingredients are listed in the US FDA inactive ingredients database, except for the novel excipient RTP004. RTP004 is a peptide with the following sequence: RKKRRQRRRG-(K)₁₅-GRKKRRQRRR (SEQ ID NO: 4). The botulinum toxin molecule is not covalently associated with R004. RT002 does not contain accessory proteins or animal-derived proteins such albumin.

TABLE 2
Composition of RT002 for Injection

| | | | Quantity per | | Quantity per | |
|---|---|---|---|---|---|---|
| | | | vial | | vial | |
| | Quality | | (50 U/vial | | (100 U/vial | |
| Ingredient | Standard | Function | formulation) | | formulation) | |
| Botulinum toxin | Internal | Active | 50 U (0.23 ng^{a}) | | 100 U (0.50 ng^{d}) | |
| Type A, 150 kD | | Ingredient | | | | |
| L-histidine | USP/Ph. Eur. | Buffer | 0.14 | mg | 0.14 | mg |
| L-histidine | Ph. Eur. | Buffer | 0.65 | mg | 0.65 | mg |
| monohydrochloride | | | | | | |
| Trehalose dihydrate | NF/Ph. Eur. | Stabilizer | 36 | mg | 36 | mg |
| Polysorbate-20 | NF/Ph. Eur. | Stabilizer | 0.1 | mg | 0.1 | mg |
| RPT004 | Internal | Stabilizer | 11.7 | μg^{b} | 11.7 | μg^{b} |

| | | | | |
|---|---|---|---|---|
| Water for Injection | USP | Processing Aid | N/A^{c} | N/A^{c} |

^{a}, Theoretical amount of toxn required for 50 U/vial drug product;
^{b}, Equivalent to 15 μg RTP004-Acetate;
^{c}, Water added in manufacturing is removed during the lyophilization step,
^{d}, Theoretical amount of toxin required for 100 U/vial drug product.

A dose escalation study using the RT002 formulation is described in the Example section of PCT Publication No. WO2018/213710 to Revance Therapeutics, Inc. This example and the entire specification of the '710 Publication is hereby incorporated by reference in its entirety.

### Example 2

This example describes a Phase 3, randomized, double-blind, placebo-controlled, parallel group, multi-center trial evaluating the efficacy and safety of a single treatment of RT002 as described in Example 1, at one of the two dosage options (125 U or 250 U), in adults with isolated (primary) cervical dystonia (CD), a movement disorder of the neck, in adults (ASPEN-1). 301 subjects with isolated (primary), moderate-to-severe cervical dystonia were enrolled at multiple sites in the United States, Canada, and Europe. Subjects were randomized (3:3:1) for injection of RT002 at 250 U (high dose, N=127), RT002 at 125 U (low dose, N=128), or placebo group (N=46).

Main eligibility criteria included subjects with a diagnosis of isolated CD meeting the full eligibility criteria. Major inclusion criterias were: adults, 18 to 80 years of age; meets diagnostic criteria for isolated CD (idiopathic, dystonic symptoms localized to the head, neck, shoulder areas) with at least moderate severity at baseline (D1), defined as Toronto Western Spasmodic Torticollis Rating Scale (TWSTRS)-total score of at least 20, with at least 15 on the TWSTRS-severity subscale, at least 3 on the TWSTRS-disability subscale, and at least 1 on the TWSTRS-pain subscale. The study population was either naïve to BoNT injection, or had a history of prior treatment with BoNT injection that was effective at controlling CD symptoms.

The RT0002 lyophilized formulation was reconstituted with sterile, non-preserved, 0.9% sodium chloride solution saline. RT002 formulations were provided in vials that were stoppered, oversealed, and stored at 2-8° C. Placebo was a sterile lyophilized product consisting of inactive ingredients without the neurotoxin supplied in single-use vials. The RT0002 and placebo were reconstituted with sterile, non-preserved 0.9% sodium chloride solution.

Investigators identified the involved muscles for injection based upon clinical representation, for example, subject's head, neck, and shoulder positions, localization of pain, and muscle hypertrophy. The volume injected in each involved muscle group was restricted to a pre-defined range, which corresponded to a specific dose per muscle for injection RT002 250 U, RT002 125 U, and placebo, respectively.

Table 1 lists injectable muscles and pre-defined volume range for injection by muscle. All solution of reconstituted study drug had to be divided and injected into the selected muscles. The sum of individual injection volume had to add up to 2.5 mL. Only unilateral injections were allowed for the sternocleidomastoid, levator scapulae, and scalenus complex. Unilateral or bilateral injections were allowed for the splenius capitis, splenius cervices, trapezius, and longissimus muscles. Volume increment for injection ranged from 0.1 mL to multiples of 0.1 mL (e.g. 0.2 mL, 0.3 mL, 0.4 mL). The number of injection sites within each muscle was determined based on the injection volume (e.g. 0.3 mL: 1-3 sites).

The primary efficacy endpoint of the Phase 3 study was an improvement in dystonia symptoms as measured by change (reduction) from baseline in Toronto Western Spasmodic Torticollis Rating Scale (TWSTRS)-Total score at four and six weeks. TWSTRS is a validated composite scale that covers different features of the cervical dystonia condition. The first part of the scale is based on the physical findings and severity of dystonia, the second part rates the patient's perceived level of disability, and the third part rates pain associated with the condition. The study protocol also features a number of secondary efficacy endpoints.

In Sum, the Study Objectives were:
To compare the efficacy of a high and low dose of RT002 for injection relative to placebo, and to each other, in adults with moderate to severe, isolated CD
To establish the duration of effect for RT002 for injection
To assess the safety and immunogenicity of RT002 for injection
   Exploratory Objectives were:
To establish effective doses for injection of muscles commonly involved in isolated CD
To assess changes in symptom burden, daily activities, psychosocial functioning, and quality of life (QOL) from patient-reported outcomes.

### The Primary Endpoint was:

improvement of dystonia, as measured by change from baseline in TWSTRS-Total score at Week 4 and 6 (TWSTRS=Toronto Western Spasmodic Torticollis Rating Scale).

The Secondary Endpoints were:
Change from baseline in TWSTRS-Total score;
Duration of effect, as assessed by the time (number of weeks) from treatment until loss of at least 80% of the peak treatment effect achieved at weeks 4 and 6, regarded as when a subject reached or exceeded their target TWSTRS-Total score;
Percentage of subjects showing at least “moderate” (a 2-point) improvement on CGIC (Clinician Global Impression of Change) at week 4 or 6; and
Percentage of subjects showing at least “moderate” (a 2-point) improvement on PGIC (Patients Global Impression of Change) at week 4 or 6; and
Safety, which included AEs, presence of serum neutralizing antibodies for BoNTA and novel excipient RT004, hematology, serum chemistry, urinalysis, ECG, vital signs, physical and neurological examinations, and clinically significant changes in pulmonary function by spirometry (FEV1/FVC).
   Exploratory Endpoints were:
Change from baseline in TWSTRS subscale scores (TWSTRS-Severity, TWSTRS-Disability, and TWSTRS-Pain) (all post-treatment timepoints)
CGIC at all post-treatment timepoints
PGIC at all post-treatment timepoints
Percentage of subjects with ≥20% decrease in the TWSTRS-total score (all post-treatment timepoints)
Percentage of subjects with ≥30% decrease in the TWSTRS-total score (all post-treatment timepoints)
Percentage of subjects in each TSQ response category at Weeks 4 and 6
Changes in symptom burden, daily activities, psychosocial functioning, and QOL based on changes in CDIP-58, WPAI, and SF-36 scores

After drug administration, subjects were followed to assess treatment response, tolerability, and safety up to 36 weeks after the injection. Subjects who experienced a treatment benefit continued the study up to 36 weeks.

The TWSTRS-total score that is consistent with loss of 80% of the peak treatment effect is called the target TWSTRS-total score. When the subject's TWSTRS-total score was the same or higher than their target TWSTRS-total score, they exited the study. Each subjects target score was calculated using their TWSTRS-total scores at baseline, week 4, and week 6.

A subject was considered to have completed the study if he/she had received the investigational product, and satisfy criteria #1 or #2 or #3 or #4 or #5:
1. Subjects with no reduction or have an increase from baseline in the average TWSTRS-total score at Weeks 4 and 6 (i.e., lack of efficacy: no improvement or worsened disease status), who complete study visits up to Week 6. Their EOS Visit is Week 6.
2. Subjects who benefit from study treatment and complete all follow-up study visits up to the time point of when their TWSTRS-total score is the same or higher than their calculated target TWSTRS score. Their EOS visit is the point when TWSTRS-total ≥target TWSTRS score.
3. Subjects who benefit from study treatment but subsequently experience significant recurrence of CD symptoms (e.g., pain) during the study before their TWSTRS-total score reaches their calculated target TWSTRS score and request retreatment, which the investigator determines is indicated based on their symptoms and neurologic examination findings. Their EOS Visit is the visit when the investigator determines that retreatment is clinically indicated.
4. Subjects who reach Week 36 and their TWSTRS-total score has not reached their target TWSTRS score, and they have not requested retreatment. Their EOS Visit is Week 36.
5. Complete protocol-specified assessments, including EOS assessments, as shown in Table 1.

### Results

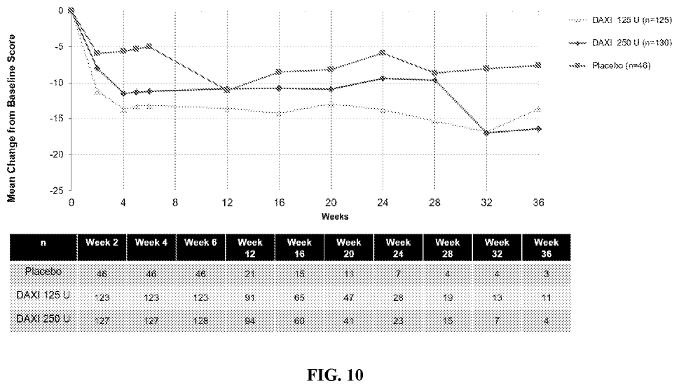

The study met its primary efficacy endpoint at both doses, demonstrating a clinically meaningful improvement in the signs and symptoms of cervical dystonia at the average of Weeks 4 and 6. Compared to placebo, subjects treated with either 125 Units or 250 Units showed a statistically significant greater change from baseline (12.7 and 10.9 respectively vs. 4.3, p<0.0001 and p=0.0006) as measured on the Toronto Western Spasmodic Torticollis Rating Scale (TWSTRS) Total Score. Nearly all subjects in this study responded to treatment and a majority were still responding to RT002 125 U at 24 weeks. The drug was generally safe and well-tolerated at both doses, with an encouraging safety profile. FIGS. 10-19 provide a summary of the data regarding the study design and results.

DURATION OF EFFECT 20 TO 24 WEEKS: The median duration of effect, defined as subjects maintaining at least 20% of the treatment benefit achieved at Weeks 4 and 6 was 24.0 and 20.3 weeks for the 125 U and 250 U doses, respectively. Median time to loss of ≥80% of peak treatment effect or request for retreatment was 20.0 and 16.4 weeks for the 125 U and 250 U doses, respectively. Median Time to loss of ≥80% of peak treatment effect or request for retreatment for Responders was 24.0 and 20.0 weeks for the 125 U and 250 U doses, respectively.

The ASPEN-1 pivotal trial demonstrates the scientific and clinical validity of a longer-acting neuromodulator with a 24 week duration, compared to 10-14 weeks observed with currently available products, XEOMIN, BOTOX, MYOBLOC, and DYSPORT. FIG. 20 provides a comparison of the duration of therapeutic effect observed with the currently available products as compared to the two RT002 doses by the amount of toxin in nanograms. The amount of the toxin in the respective products in nanograms was obtained from Field, et al. AbobotulinumtoxinA (Dysport®), OnabotulinumtoxinA (Botox®), and IncobotulinumtoxinA (Xeomin®) Neurotoxin Content and Potential Implications for Duration of Response in Patients, Toxins 2018, 10(12), 535.

POSITIVE EFFICACY RESULTS: The trial's 4-week primary efficacy measurement was the improvement in signs and symptoms of cervical dystonia as determined by reduction of the TWSTRS-Total score from baseline. The average improvement from baseline in the TWSTRS total score at weeks 4 and 6 was 31% and 27% (12.7 and 10.9 points), respectively, for RT002 125 U and RT002 250 U (both p<0.001 versus placebo).

TWSTRS Severity, Disability, and Pain Subscales showed a similar magnitude of improvement from baseline: 30-32% for 125 U and 24-26% for 250 U v. 11-12% for placebo (p<0.02 for all comparisons v. placebo).

Both Clinician Global Impression of Change (CGIC) and Patient Global Impression of Change (PGIC) demonstrated improvement consistent with the primary endpoint in TWSTRS Total Score. 77-78% of subjects experienced improvement at Weeks 4 or 6 in their CD symptoms as assessed by CGIC 71-73% of subjects experienced improvement at Weeks 4 or 6 in their CD symptoms as assessed by PGIC.

GENERALLY SAFE AND WELL-TOLERATED: RT002 at both 125 U and 250 U doses was generally well-tolerated with no trend toward increasing adverse events with increased dose. Treatment-Related Adverse Events occurred in 17.4%, 29.1% and 24.2% of subjects in the placebo, 125 U and 250 U dose groups, respectively. Most frequently reported events in the RT002 125 U and 250 U dose groups were injection site pain (7.9% and 4.7%), headache (2.2% and 4.7%) and injection site pain (4.7% and 2.3%), respectively. Dysphagia occurred in 1.6% and 3.9% of subjects in the 125 U and 250 U dose groups, respectively. All cases of dysphagia were mild in severity. Majority of Treatment Related AE's were mild or moderate in severity. A tabular summary of the adverse events is provided below in Tables 3 and 4.

TABLE 3

| | | RT002 | RT002 | |
|---|---|---|---|---|
| AE Summary | Placebo | 125 U | 250 U | Total |
| n (%) # of Events | (N = 46) | (N = 127) | (N = 128) | N = 301) |
| Subjects with any TEAEs | 18 (39.1) 34 | 74 (58.3) 148 | 64 (50.0) 134 | 156 (51.8) 316 |
| Subjects with any Treatment-Related | 8 (17.4) 11 | 37 (29.1) 54 | 31 (24.2) 49 | 76 (25.2) 114 |
| TEAEs | | | | |
| Mild | 8 (17.4) | 34 (26.8) | 29 (22.7) | 71 (23.6) |
| Moderate | 0 | 2 (1.6) | 2 (1.6) | 4 (1.3) |
| Severe* | 0 | 1 (0.8) | 0 | 1 (0.3) |
| Subjects with any Serious TEAEs** | 0 | 5 (3.9) 5 | 3 (2.3) 4 | 8 (2.7) 9 |
| Subjects with any TEAEs Leading to | 0 | 1 (0.8) 1 | 0 | 1 (0.3) 1 |
| Death | | | | |
| Subjects with any TEAEs Leading to | 0 | 0 | 1 (0.8) 1 | 1 (0.3) 1 |
| Study Discontinuation | | | | |

TABLE 4

| | | RT002 | RT002 | |
|---|---|---|---|---|
| AE Summary | Placebo | 125 U | 250 U | Total |
| n (%) | (N = 46) | (N = 127) | (N = 128) | (N = 301) |
| Subjects with any | 8 (17.4) | 37 (29.1) | 31 (24.2) | 76 (25.2) |
| Treatment-Related TEAE | | | | |
| Injection site pain | 2 (4.3) | 10 (7.9) | 6 (4.7) | 18 (6.0) |
| Headache | 1 (2.2) | 6 (4.7) | 6 (4.7) | 13 (4.3) |
| Injection site erythema | 1 (2.2) | 6 (4.7) | 3 (2.3) | 10 (3.3) |
| Muscular weakness | 0 | 6 (4.7) | 3 (2.3) | 9 (3.0) |
| Musculoskeletal pain | 0 | 3 (2.4) | 4 (3.1) | 7 (2.3) |
| Dysphagia | 0 | 2 (1.6) | 5 (3.9) | 7 (2.3) |

In comparison to competitor formulations, the study resulted in a lower incidence of dysphagia, musculoskeletal pain, and muscular weakness for both the 125 U and 250 U doses of RT002. For example, Table 5 below provides a comparison of the rate of dysphagia reported on the respective product's prescribing information (Year 2020).

TABLE 5

| Product, dose | Dysphagia Rate |
|---|---|
| RT002, 125 U | 1.6% |
| RT002, 250 U | 3.9% |
| Xeomin, 120 U | 13% |
| Dysport, 500 U | 15% |
| Xeomin, 240 U | 18% |
| Botox, 236 U | 19% |

No new or unexpected Treatment Emergent Adverse Events were observed. Incidence of Treatment-related AE's observed was similar or lower compared with prior BoNT studies in CD. No Serious Adverse Events related to study treatment were reported.

The Safety Summary of the study: Both RT002 125 U and RT002 250 appeared to be generally safe and well tolerated through Week 24, with no trend toward increasing adverse events with increased dose.

Treatment-Related Adverse Events occurred in 17.4%, 29.1% and 24.2% of subjects in the placebo, 125 U and 250 U dose groups, respectively.
Most frequently reported: Injection site pain (total 6%), headache (total 4.3%), injection site erythema (total 3.3%), muscular weakness (total 3%), musculoskeletal pain (total 2.3%), and dysphagia (total 2.3%). Most frequently reported events in the RT002 125 U and RT002 250 U dose groups were injection site pain (7.9% and 4.7%) and headache (4.7% and 4.7%), respectively.
All Treatment-Related TEAE's were mild or moderate in severity. No Serious AEs were reported.

One subject had a TEAE with outcome death, which wasn't considered treatment-related. No AEs were reported while the subject was on study and all chemistry and hematology results were normal. At baseline, the C-SSRS was negative for any suicidal ideation or behavior.

Dysphagia occurred in 1.6% and 3.9% of subjects in the 125 U and 250 U dose groups, respectively. All cases of dysphagia were mild in severity.

No new or unexpected TEAEs were observed. Incidence of Treatment-related AE's observed was similar or lower compared with prior BoNT studies in CD. Trials of other BoNTA products approved to treat CD have dysphagia rates ranging from 13-39%. Includes BOTOX, Dysport and Xeomin. Data as reported in product label.

### The Efficacy Summary of the Study:

Duration of Effect: The median duration of effect (95%, CI), defined as the time in weeks from treatment until loss of at least 80% of the peak treatment effect, was 24 weeks (20.3, 29.1) for 125 U dose and 20.3 weeks (16.7, 24.0) for 250 U dose. The peak treatment effect is defined as the average change from baseline at week 4 and week 6 in the TWSTRS total score.

Improvement in CD Signs and Symptoms: A clinically significant reduction from baseline in the TWSTRS Total Score at week 4 and week 6 was 12.7 and 10.9 points (31% and 27%) for RT002 125 U and 250 U (both p<0.001 v. placebo). TWSTRS Severity, Disability, and Pain Subscales showed a similar magnitude of improvement from baseline: 30-32% for 125 U and 24-26% for 250 U v. 11-12% for placebo (p<0.02 for all comparisons v. placebo).
The improvement from baseline peaked at 31% (125 U) and 27% (250 U) at Week 4, with the majority of this treatment benefit maintained through Week 24.

Global Impression of Change Response Rate: Both Clinician Global Impression of Change (CGIC) and Patient Global Impression of Change (PGIC) demonstrated improvement consistent with the primary endpoint in TWSTRS Total Score. 77-78% of subjects experienced improvement at Weeks 4 or 6 in their CD symptoms as assessed by CGIC. 71-73% of subjects experienced improvement at Weeks 4 or 6 in their CD symptoms as assessed by PGIC.

CONCLUSIONS: ASPEN-1 demonstrated that RT002 for injection, at either 125 U or 250 U, was an effective and generally well-tolerated treatment for reducing the signs and symptoms for CD for up to 24 weeks.

RT002 met primary and all secondary endpoints for reduction of signs and symptoms associated with CD. Highly statistically significant results achieved on TWSTRS-Total Score primary endpoint at Weeks 4 and 6. Median duration for time to reach Target TWSTRS Score was 24 weeks for 125 U dose and 20.3 weeks for 250 U dose/Superior improvement observed on CGIC and PGIC compared with placebo at Weeks 4 and 6. 150 kD, Type A BoNT appeared to be generally safe and well-tolerated with safety profile similar to other BoNT products for the treatment of CD.

These positive ASPEN results reinforce the findings from the previous studies with 150 kD, Type A BoNT as a highly differentiated neuromodulator. The ASPEN-1 pivotal trial demonstrates the scientific and clinical validity of a longer-acting neuromodulator with a 24 week duration, compared to 10-14 weeks observed with currently available products.

### Example 3

This example is a Phase 3, open-label, multi-center trial to evaluate the long-term safety, efficacy, and immunogenicity of repeat (up to four) treatments of RT002 at doses of 125 U, 200 U, 250 U, and 300 U in adults with isolated cervical dystonia (CD) (ASPEN-OLS). Approximately 350 subjects with isolated (primary), moderate-to-severe cervical dystonia will be recruited from study centers in the United States, Canada, and Europe, who were enrolled in ASPEN-1 (Example 2). These subjects from Study ASPEN-1 include:
Those with no reduction or increase from baseline in the average TWSTRS-total score at Weeks 4 and 6 (i.e., no improvement or worsened disease), and the investigator agreed that there was a need for retreatment based on the subject's symptoms and neurologic exam findings
Those who benefited from study treatment and completed follow-up study visits up to the time point of when their TWSTRS-total score reached/exceeded their target TWSTRS
Those who benefited from study treatment but subsequently experienced significant recurrence of CD symptoms (e.g. pain) during the study before their TWSTRS-total score reached their target TWSTRS score and requested retreatment, which the investigator determined was warranted based on the subject's symptoms and neurologic exam findings
Those who completed study visits up to Week 36 and their TWSTRS-total score never reached their target TWSTRS score. The investigator determined that these subjects can be followed in the OLS until their TWSTRS-total score is the same or higher than their target TWSTRS score or until they request retreatment, which the investigator determined was clinically indicated

Main eligibility criteria included subjects with a diagnosis of isolated CD meeting the full eligibility criteria. Major inclusion criterias are: adults, 18 to 80 years of age; meets diagnostic criteria for isolated CD (idiopathic, dystonic symptoms localized to the head, neck, shoulder areas) with at least moderate severity at baseline (D1), defined as Toronto Western Spasmodic Torticollis Rating Scale (TWSTRS)-total score of at least 20, with at least 15 on the TWSTRS-severity subscale, at least 3 on the TWSTRS-disability subscale, and at least 1 on the TWSTRS-pain subscale. TWSTRS-subscale criteria are only applicable for subjects not previously enrolled in Study Protocol ASPEN-1.

RT002 125 U and 250 U will be provided in vials that are stored at 2-8° C. Placebo is a sterile lyophilized product consisting of inactive ingredients without the neurotoxin supplied in single-use vials to be reconstituted with sterile, non-preserved 0.9% sodium chloride solution.

RT002 will be administered as multiple IM injections in affected muscles, with muscles injected according to pattern of involvement. The initial dose (125 U or 250 U) in Treatment Cycle 1 will be determined by the investigator based on the criteria described above. In subsequent treatment cycles, the dose can remain the same or can be titrated up or down by one pre-defined dose-step per subsequent treatment cycle, based on the treatment response from the prior cycle as determined by the Investigator. For Treatment Cycles 2 to 4, the dose can be 125 U (minimum dose), 200 U, 250 U, or 300 U (maximum dose).

Investigators identified the involved muscles for injection based upon clinical representation, for example, subject's head, neck, and shoulder positions, localization of pain, and muscle hypertrophy). The volume injected in each involved muscle group was restricted to a pre-defined range, which corresponded to a specific dose per muscle for RT002 250 U, RT002 125 U, and placebo, respectively.

Table 6 lists injectable muscles and pre-defined volume range for injection by muscle. The investigator should identify the muscles for injection among those listed based on the subject's head, neck, and shoulder position, localization of pain, and muscle hypertrophy. The pre-defined injection dose range by muscle for the four injection doses are listed in Table 6. All doses for injection (125 U, 200 U, 250 U, and 300 U) are reconstituted with non-preserved, 0.9% sodium chloride solution for injection.

### Mandatory Injection Parameters:

All of the solution of reconstituted study drug must be divided and injected into the selected muscles
For each selected muscle, the injection dose must be within the pre-defined dose range for the muscle
DO NOT inject any other muscles than those identified in Table 3
Use of EMG, ultrasonography, or other imaging modalities is optional
Only unilateral injections can be performed for the sternocleidomastoid, levator scapulae, and scalenus complex (if any of these muscles are selected for injection).
Unilateral or bilateral injections can be performed for the splenius capitis, splenius cervices, trapezius, and longissimus (if any of these muscles are selected for injection

TABLE 6
Injectable Muscles and Pre-defined Injection Dose per Muscle

| | Dilution | RT002 125 U | RT002 200 U | RT002 250 U | RT002 300 U |
|---|---|---|---|---|---|
| | Injection | 5 U/ 0.1 mL | 8 U/ 0.1 mL | 10 U/ 0.1 mL | 12 U/ 0.1 mL |
| | Volume | Injection | Injection | Injection | Injection |
| | Range | Dose Range | Dose Range | Dose Range | Dose Range |
| Injectable Muscles | (mL) | (Units) | (Units | (Units | (Units |
| Sternocleidomastoid | 0.2-0.5 | 10-25 | 16-40 | 20-50 | 24-60 |
| Levator Scapulae | 0.2-0.6 | 10-30 | 16-48 | 20-60 | 24-72 |
| Scalenus Complex | 0.2-0.3 | 10-15 | 16-24 | 20-30 | 24-36 |
| Splenius Capitis | 0.2-1.0 | 10-50 | 16-80 | 20-100 | 24-120 |
| Splenius Cervices | 0.2-1.0 | 10-50 | 16-80 | 20-100 | 24-120 |
| Trapezius | 0.3-0.8 | 15-40 | 24-64 | 30-80 | 36-96 |
| Longissimus | 0.2-0.6 | 10-30 | 16-48 | 20-60 | 24-72 |

*

The primary efficacy endpoint of the Phase 3 study is to evaluate long-term safety of multiple continuous treatments of RT002 by injection. The study protocol also features a number of secondary efficacy endpoints.

### In Sum, the Study Objectives are:

To evaluate long-term safety of multiple continuous treatments of RT002 for injection (primary objective)
To assess immunogenicity to BoNT TypeA and RTP004 after multiple treatments of RT002 by injection (primary objective)
To evaluate long-term efficacy of multiple continuous treatments of RT002 by injection (secondary objective)
To establish the inter-treatment time interval or duration of effect (secondary objective)
To evaluate changes in symptom burden, daily activities, and psychosocial functioning after multiple continuous treatments of RT002 by injection (secondary objective)

### Exploratory Objectives are:

To evaluate changes in treatment satisfaction after multiple continuous treatments of RT002 by injection
To evaluate changes in health-related quality of life (QOL) after multiple continuous treatments of RT002 by injection

### The Primary Endpoints are:

Dose- and cycle-specific incidence of drug-related AEs
Dose- and cycle-specific incidence of study drug discontinuation due to drug-related AEs
Dose- and cycle-specific of treatment-emergent immunogenicity

### The Secondary Endpoints are:

Inter-treatment time interval or duration of effect
The dose- and cycle-specific average of the change in the TWSTRS-total score at Weeks 4 and 6 of each treatment cycle
Percentage of subjects with at least “moderate” (a 2-point) improvement on CGIC at Week 4 or Week 6 of the treatment cycle
Percentage of subjects with at least “moderate” (a 2-point) improvement on PGIC at Week 4 or Week 6 of the treatment cycle
Changes in quality of life measures based on the CDIP-58
   Exploratory Endpoints were:
The dose- and cycle-specific incidence of AEs associated with distant spread of toxin
The dose- and cycle-specific average of the change from baseline in TWSTRS-Severity, -Disability, and -Pain subscale scores at Weeks 4 and 6 of each treatment
The dose- and cycle-specific averages of the change from baseline in the TWSTRS-total score at each visit
CGIC at all post-treatment timepoints
PGIC at all post-treatment timepoints
Percentage of subjects with ≥20% decrease in the TWSTRS-total score (all post-treatment timepoints)
Percentage of subjects with ≥30% decrease in the TWSTRS-total score (all post-treatment timepoints)
The cycle-specific Treatment Satisfaction Questionnaire (TSQ) score
The cycle-specific changes in Work Productivity and Activity Impairment (WPAI) score
The cycle-specific change in Short Form-36 (SF-36) score

The TWSTRS-total score that is consistent with loss of 80% of the peak treatment effect is called the target TWSTRS-total score. Each subjects target score is calculated using their TWSTRS-total scores at baseline, week 4, and week 6 in treatment cycle 1. The next treatment cycle cannot occur sooner than 12 weeks after the last treatment. Subjects with treatment benefit may receive up to 4 treatments over a 52-week study period. Subjects with treatment benefit sustained for longer than 12 weeks between treatment cycles may receive 1 to 3 treatments over a 52-weeks study period. In treatment cycles 2 to 4, the investigator can choose to administer the same dose of RT002 by injection (125 U or 250 U) the subject received in cycle 1, or increase or decrease the dose of RT002 by for injection to be administered by one pre-defined dose-step per subsequent treatment cycle based on the subject's treatment response in the prior cycle. Two additional doses of v injection are introduced starting in Treatment Cycle 2: 200 U and 300 U of v injection. The dose to be injected into each involved muscle is restricted to a pre-defined dose range by muscle. Table 3 lists pre-defined dose ranges for injection by muscle and total dose of RT002 by injection. In Treatment Cycle 2-4 subjects may receive a minimum of 125 U or a maximum of 300 U per

It is understood that the following examples and embodiments described herein are for illustrative purposes and that various modifications or changes in light thereof will be suggested to persons skilled in the art and are to be included within the spirit and purview of this application and scope of the appended claims.

All publications, patents, and published patent applications cited herein are hereby incorporated by reference in their entireties for all purposes.

1. A method of treating cervical dystonia in an individual, the method comprising:
administering by injection a treatment dose of a sterile injectable composition into one or more of the muscles causing the cervical dystonia in the individual in need of treatment to achieve the therapeutic effect;
wherein the composition comprises a pharmaceutically acceptable diluent suitable for injection and a botulinum toxin of serotype A and 150 kD;
wherein the total treatment dose of botulinum toxin component administered to the individual is 100 U to 500 U;
wherein the composition comprises a positively charged carrier having a positively charged backbone of polylysine having covalently attached to each end a positively charged efficiency groups having an amino acid sequence selected from (gly)ₚ-RGRDDRRQRRR-(gly)_{q} (SEQ ID NO: 1), (gly)ₚ-YGRKKRRORRR-(gly)_{q} (SEQ ID NO: 2) or (gly)ₚ-RKKRRQRRR-(gly)_{q} (SEQ ID NO: 3), wherein the subscripts p and q are each independently an integer of from 0 to 20;
wherein the one or more muscles comprise at least one of sternocleidomastoid levator scapulae, and scalenus complex and wherein the sternocleidomastoid, levator scapulae, and scalenus complex are only unilaterally injected, and
wherein the therapeutic effect is associated with a risk of adverse events, risk of dysphagia, risk of musculoskeletal pain, and/or risk of muscular weakness that is below 15%.

2. (canceled)

3. The method of claim 1, wherein the one or more muscles comprise sternocleidomastoid, levator scapulae, and scalenus complex.

4. The method according to claim 1, wherein the dose range for the sternocleidomastoid, levator scapulae, and scalenus complex is set forth in Table 1 or Table 6.

5. A method of treating cervical dystonia in an individual, the method comprising:
administering by injection a treatment dose of a sterile injectable composition into one or more of the muscles causing the cervical dystonia in the individual in need of treatment to achieve the therapeutic effect;
wherein the composition comprises a pharmaceutically acceptable diluent suitable for injection; and a botulinum toxin of serotype A and 150 kD;
wherein the total treatment dose of botulinum toxin component administered to the individual is 100 U to 500 U;
wherein the composition comprises a positively charged carrier having a positively charged backbone of polylysine having covalently attached to each end a positively charged efficiency group having an amino acid sequence selected from (gly)ₚ-RGRDDRRQRRR-(gly)_{q} (SEQ ID NO: 1), (gly)ₚ-YGRKKRRORRR-(gly)_{q} (SEQ ID NO: 2) or (gly)ₚ-RKKRRQRRR-(gly)_{q} (SEQ ID NO: 3), wherein the subscripts p and q are each independently an integer of from 0 to 20; and
wherein the one or more muscles are selected from a group comprising or consisting of splenius capitis, splenius cervices, trapezius, longissimus, sternocleidomastoid, levator scapulae, and scalenus complex, wherein the dose range for each of the one or more muscles is set forth in Table 1 or Table 6; and
wherein the therapeutic effect is associated with a risk of adverse events, risk of dysphagia, risk of musculoskeletal pain, and/or risk of muscular weakness that is below 15%.

6. (canceled)

7. The method of claim 5, wherein the sternocleidomastoid, levator scapulae, and scalenus complex are only unilaterally injected.

8. The method according to claim 1, wherein the risk of adverse events is less than 12%, 10%, 8%, 7%, 5%, or 3%.

9. (canceled)

10. The method of claim 1, wherein (i) the subscripts p and q are each independently an integer of from 0 to 8; or (ii) are each independently an integer of from 2 to 5.

11. (canceled)

12. The method of claim 10, wherein the positively charged carrier has the amino acid sequence RKKRRQRRRG-(K)₁₅-GRKKRRQRRR (SEQ ID NO: 4).

13. (canceled)

14. (canceled)

15. The method according to claim 1, wherein the treatment dose of botulinum toxin is administered to the individual in an amount of about 100 U to 200 U or 125 U.

16. The method according to claim 1, wherein the treatment dose of botulinum toxin is administered to the individual in an amount of about 200 U to 300 U or 250 U.

17. The method according to claim 1, wherein the treatment dose of botulinum toxin is administered to the individual in an amount of 300 U to 450 U or 300 U.

18. (canceled)

19. (canceled)

20. (canceled)

21. The method according to claim 1, wherein the duration of treatment effect comprises at least 16 weeks.

22. The method according to claim 1, wherein the duration of treatment effect comprises at least 20 weeks.

23. The method according to claim 1, wherein the duration of treatment effect comprises at least 22 weeks.

24. The method according to claim 1, wherein the duration of treatment effect comprises at least 24 weeks

25. The method according to claim 1, wherein the duration of treatment effect comprises at least 26 weeks

26. (canceled)

27. (canceled)

28. The method according to claim 1, wherein the risk of dysphagia from the treatment is less than 12%, 10%, 8%, 7%, 5%, or 3%.

29. The method according to claim 1, wherein the risk of musculoskeletal pain from the treatment is less than 12%, 10%, 8%, 7%, 5%, or 3%.

30. The method according to claim 1, wherein the risk of muscular weakness from the treatment is less than 12%, 10%, 8%, 7%, 5%, or 3%.


SEQUENCE LISTING (10 sequences)

SEQ ID NO: 1 - protein, 51 aa, Artificial Sequence
  Description of Artificial Sequence Synthetic polypeptide
  MISC_FEATURE (1)..(20): This region may encompass 0-20 ′Gly′ residues wherein some positions may be absent
  MISC_FEATURE (32)..(51): This region may encompass 0-20 ′Gly′ residues wherein some positions may be absent
  See specification as filed for detailed description of substitutions and preferred embodiments
GGGGGGGGGGGGGGGGGGGGRGRDDRRQRRRGGGGGGGGGGGGGGGGGGGG

SEQ ID NO: 2 - protein, 51 aa, Artificial Sequence
  Description of Artificial Sequence Synthetic polypeptide
  MISC_FEATURE (1)..(20): This region may encompass 0-20 ′Gly′ residues wherein some positions may be absent
  MISC_FEATURE (32)..(51): This region may encompass 0-20 ′Gly′ residues wherein some positions may be absent
  See specification as filed for detailed description of substitutions and preferred embodiments
GGGGGGGGGGGGGGGGGGGGYGRKKRRQRRRGGGGGGGGGGGGGGGGGGGG

SEQ ID NO: 3 - protein, 49 aa, Artificial Sequence
  Description of Artificial Sequence Synthetic polypeptide
  MISC_FEATURE (1)..(20): This region may encompass 0-20 ′Gly′ residues wherein some positions may be absent
  MISC_FEATURE (30)..(49): This region may encompass 0-20 ′Gly′ residues wherein some positions may be absent
  See specification as filed for detailed description of substitutions and preferred embodiments
GGGGGGGGGGGGGGGGGGGGRKKRRQRRRGGGGGGGGGGGGGGGGGGGG

SEQ ID NO: 4 - protein, 35 aa, Artificial Sequence
  Description of Artificial Sequence Synthetic polypeptide
RKKRRQRRRGKKKKKKKKKKKKKKKGRKKRRQRRR

SEQ ID NO: 5 - protein, 45 aa, Artificial Sequence
  Description of Artificial Sequence Synthetic polypeptide
  MISC_FEATURE (1)..(20): This region may encompass 0-20 ′Gly′ residues wherein some positions may be absent
  MISC_FEATURE (21)..(45): This region may encompass 5, 7, 9, 11, 13, 15, 17, 21, 23 or 25 ′Arg′ residues wherein some positions may be absent
  See specification as filed for detailed description of substitutions and preferred embodiments
GGGGGGGGGGGGGGGGGGGGRRRRRRRRRRRRRRRRRRRRRRRRR

SEQ ID NO: 6 - protein, 19 aa, Drosophila sp.
SGRQIKIWFQNRRMKWKKC

SEQ ID NO: 7 - protein, 39 aa, Artificial Sequence
  Description of Artificial Sequence Synthetic polypeptide
YGRKKRRQRRRGKKKKKKKKKKKKKKKGYGRKKRRQRRR

SEQ ID NO: 8 - protein, 39 aa, Artificial Sequence
  Description of Artificial Sequence Synthetic polypeptide
RGRDDRRQRRRGKKKKKKKKKKKKKKKGRGRDDRRQRRR

SEQ ID NO: 9 - protein, 20 aa, Artificial Sequence
  Description of Artificial Sequence Synthetic peptide
  MISC_FEATURE (1)..(20): This sequence may encompass 10-20 ′Lys′ residues wherein some positions may be absent
  See specification as filed for detailed description of substitutions and preferred embodiments
KKKKKKKKKKKKKKKKKKKK

SEQ ID NO: 10 - protein, 10 aa, Artificial Sequence
  Description of Artificial Sequence Synthetic peptide
GGGRRRRRRR